(19) United States
(12) Patent Application Publication
(10) Pub. No.: US 2024/0132956 A1
(43) Pub. Date: Apr. 25, 2024

(54) NUCLEIC ACID SEQUENCING COMPONENTS INCLUDING A GLYCOLIPID BI-LAYER

(71) Applicant: ILLUMINA, INC., San Diego, CA (US)

(72) Inventors: Justin Robbins, San Diego, CA (US); Mathieu Lessard-Viger, San Diego, CA (US)

(21) Appl. No.: 18/476,151

(22) Filed: Sep. 27, 2023

Related U.S. Application Data
(60) Provisional application No. 63/377,690, filed on Sep. 29, 2022.

(51) Int. Cl. C12Q 1/6874; B01L 3/00

(52) U.S. Cl. CPC C12Q 1/6874; B01L 3/502761; B01L 2200/0647; B01L 2200/12; B01L 2300/0896; B01L 2300/12

(57) ABSTRACT

An example of a nucleic acid sequencing component includes a support. A glycolipid bi-layer is attached to at least a portion of the support. First and second primers are respectively attached to the glycolipid bi-layer. In one example, the support is a substrate of a flow cell. In another example, the support is a core nanostructure that can be introduced into a flow cell.

### CROSS REFERENCE TO RELATED APPLICATION

This application claims the benefit of U.S. Provisional Application Ser. No. 63/377,690, filed Sep. 29, 2022, the contents of which is incorporated by reference herein in its entirety.

### REFERENCE TO SEQUENCE LISTING

The instant application contains a Sequence Listing which has been submitted electronically in XML file format and is hereby incorporated by reference in its entirety. Said XML copy, created on Sep. 1, 2023, is named ILI239B_IP-2343-US_Sequence_Listing.xml and is 12,254 bytes in size.

### BACKGROUND

Genetic analysis, and in particular nucleic acid sequencing, is taking on increasing importance in modern society, as it has proven useful in a variety of applications. As examples, genetic analysis has been used in predicting a person's risk of contracting some diseases (diagnostics), in determining the probability of therapeutic benefit versus the risk of side effects for a person considering certain treatments (prognostics), and in identifying missing persons, perpetrators of crimes, victims of crimes and casualties of war (forensics). Due to the rapidly increasing demand for reliable genetic information related to an organism, a disease, or an individual, improvements in the throughput of the analysis methods are constantly being investigated.

### SUMMARY

Disclosed herein are different examples of nucleic acid sequencing components that include a glycolipid bi-layer and primers attached to the glycolipid bi-layer. The glycolipid bi-layer introduces a biological environment to the sequencing component that can enhance the function(s) of the enzyme(s) (e.g., polymerase(s)) used in a sequencing by synthesis operation. The glycolipid bi-layer supports enzymatic nucleic acid template replication, and thus can enhance the amplification kinetics during cluster generation. Moreover, the glycolipid bi-layer enhances polymerase activity by i) enriching the surface with labeled nucleotides and metal co-factors used in nucleic acid sequencing and ii) sequestering waste products (inorganic pyrophosphatase (PR)) of the synthesis reaction. As such, the glycolipid bi-layer can enhance the polymerase synthesis activity and the forward reaction kinetics. An enhanced rate of kinetics during sequencing by synthesis decreases the overall workflow turnaround times.

Additionally, the glycolipid bi-layer can be disrupted, enabling a portion of the glycolipid bi-layer to be removed following a sequencing operation. Sequestered waste products can be removed with the disrupted portion of the glycolipid bi-layer. A fresh glycolipid bi-layer can be generated for use in a subsequent sequencing operation.

### BRIEF DESCRIPTION OF THE FIGURES

Features of examples of the present disclosure will become apparent by reference to the following detailed description and drawings, in which like reference numerals correspond to similar, though perhaps not identical, components. For the sake of brevity, reference numerals or features having a previously described function may or may not be described in connection with other drawings in which they appear.

FIG. 1A is a schematic, cross-sectional view of a flow cell substrate including a glycolipid bi-layer in a lane;

FIG. 1B is a schematic, cross-sectional view of a flow cell substrate including a glycolipid bi-layer in depressions;

FIG. 1C is a schematic view of a functionalized nanostructure including a glycolipid bi-layer at the surface;

FIG. 2 is a schematic flow diagram illustrating one example of a method for making a nucleic acid sequencing component;

FIG. 3 is a schematic flow diagram illustrating another example of a method for making a nucleic acid sequencing component;

FIG. 4 is a schematic flow diagram illustrating yet another example of a method for making a nucleic acid sequencing component;

FIG. 5A is a schematic, cross-sectional view of one example of an enclosed flow cell including capture sites;

FIG. 5B is a schematic, cross-sectional view of another example of an enclosed flow cell including capture sites; and

FIG. 6 is a schematic flow diagram illustrating an example of a method for disrupting a glycolipid bi-layer after sequencing and regenerating a fresh glycolipid bi-layer.

### DETAILED DESCRIPTION

Each of the nucleic acid sequencing components disclosed herein is functionalized with a biological environment that enhances the function(s) of the enzyme(s) (e.g., polymerase(s)) used in a sequencing by synthesis operation. Enhanced enzyme function can increase the rate of kinetics during one or more reactions of the sequencing by synthesis operation, including cluster generation and nascent strand formation. The biological environment includes a glycolipid bi-layer and primers attached to the glycolipid bi-layer.

### Definitions

It is to be understood that terms used herein will take on their ordinary meaning in the relevant art unless specified otherwise. Several terms used herein and their meanings are set forth below.

The singular forms “a”, “an”, and “the” include plural referents unless the context clearly dictates otherwise.

The terms comprising, including, containing and various forms of these terms are synonymous with each other and are meant to be equally broad.

The terms top, bottom, lower, upper, on, etc. are used herein to describe the nucleic acid sequencing component and/or the various sub-components of the nucleic acid sequencing component. It is to be understood that these directional terms are not meant to imply a specific orientation, but are used to designate relative orientation between sub-components. The use of directional terms should not be interpreted to limit the examples disclosed herein to any specific orientation(s).

The terms first, second, etc. also are not meant to imply a specific orientation or order, but rather are used to distinguish one component or sub-component from another.

It is to be understood that the ranges provided herein include the stated range and any value or sub-range within the stated range, as if such values or sub-ranges were explicitly recited. For example, a range of about 400 nm to about 1 μm (1000 nm), should be interpreted to include not only the explicitly recited limits of about 400 nm to about 1 μm, but also to include individual values, such as about 708 nm, about 945.5 nm, etc., and sub-ranges, such as from about 425 nm to about 825 nm, from about 550 nm to about 940 nm, etc. Furthermore, when “about” and/or “substantially” are/is utilized to describe a value, they are meant to encompass minor variations (up to +/−10%) from the stated value.

An “acrylamide monomer” is a monomer with the structure

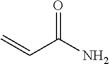

or a monomer including an acrylamide group. Examples of the monomer including an acrylamide group include azido acetamido pentyl acrylamide:

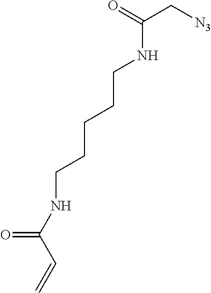 [SMILES: C=CC(=O)NCCCCCNC(=O)CN=[N+]=[N-]]

### and N-isopropylacrylamide:

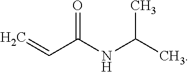

Other acrylamide monomers may be used.

An “aldehyde,” as used herein, is an organic compound containing a functional group with the structure —CHO, which includes a carbonyl center (i.e., a carbon double-bonded to oxygen) with the carbon atom also bonded to hydrogen and an R group, such as an alkyl or other side chain. The general structure of an aldehyde is:

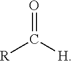

As used herein, “alkyl” refers to a straight or branched hydrocarbon chain that is fully saturated (i.e., contains no double or triple bonds). The alkyl group may have 1 to 20 carbon atoms. Example alkyl groups include methyl, ethyl, propyl, isopropyl, butyl, isobutyl, tertiary butyl, pentyl, hexyl, and the like. As an example, the designation “C1-4 alkyl” indicates that there are one to four carbon atoms in the alkyl chain, i.e., the alkyl chain is selected from the group consisting of methyl, ethyl, propyl, iso-propyl, n-butyl, isobutyl, sec-butyl, and t-butyl.

As used herein, “alkenyl” refers to a straight or branched hydrocarbon chain containing one or more double bonds. The alkenyl group may have 2 to 20 carbon atoms. Example alkenyl groups include ethenyl, propenyl, butenyl, pentenyl, hexenyl, and the like.

As used herein, “alkyne” or “alkynyl” refers to a straight or branched hydrocarbon chain containing one or more triple bonds. The alkynyl group may have 2 to 20 carbon atoms.

As used herein, “aryl” refers to an aromatic ring or ring system (i.e., two or more fused rings that share two adjacent carbon atoms) containing only carbon in the ring backbone. When the aryl is a ring system, every ring in the system is aromatic. The aryl group may have 6 to 18 carbon atoms. Examples of aryl groups include phenyl, naphthyl, azulenyl, and anthracenyl.

An “amino” functional group refers to an —NRₐR_{b} group, where Rₐ and R_{b} are each independently selected from hydrogen

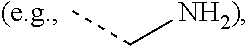

C1-6 alkyl, C2-6 alkenyl, C2-6 alkynyl, C3-7 carbocyclyl, C6-10 aryl, 5-10 membered heteroaryl, and 5-10 membered heterocyclyl, as defined herein.

As used herein, the term “attached” refers to the state of two things being joined, fastened, adhered, connected or bound to each other, either directly or indirectly. With indirect attachment, an intermediate is present between the two things that are attached (e.g., a hydrogel may be positioned between a glycolipid bi-layer and a support so that the glycolipid bi-layer is indirectly attached to the support). The attachment may be a bond that is covalent or non-covalent. A covalent bond is characterized by the sharing of pairs of electrons between atoms. A non-covalent bond is a physical bond that does not involve the sharing of pairs of electrons and can include, for example, hydrogen bonds, ionic bonds, van der Waals forces, hydrophilic interactions and hydrophobic interactions.

An “azide” or “azido” functional group refers to —N₃.

A “capture site”, as used herein, refers to portion of a flow cell substrate having been modified, chemically, magnetically or electrostatically, that allows for anchoring of one example of the nucleic acid sequencing component. In an example, the capture site may include a chemical capture agent, a magnetic capture agent, or an electrostatic capture agent.

As used herein, “carbocycle” means a non-aromatic cyclic ring or ring system containing only carbon atoms in the ring system backbone. When the carbocycle is a ring system, two or more rings may be joined together in a fused, bridged or spiro-connected fashion. Carbocycles may have any degree of saturation, provided that at least one ring in a ring system is not aromatic. Thus, carbocycles include cycloalkyls, cycloalkenyls, and cycloalkynyls. The carbocycle group may have 3 to 20 carbon atoms. Examples of carbocycle rings include cyclopropyl, cyclobutyl, cyclopentyl, cyclohexyl, cyclohexenyl, 2,3-dihydro-indene, bicyclo[2.2.2]octanyl, adamantyl, and spiro[ 4.4]nonanyl.

As used herein, the term “carboxylic acid” or “carboxyl” as used herein refers to —COOH.

A “chemical capture agent” is a material, molecule or moiety that is capable of anchoring to a functional agent of one example of the nucleic acid sequencing component via a chemical mechanism. One example chemical capture agent includes a capture nucleic acid (e.g., a capture oligonucleotide) that is complementary to at least a portion of a target nucleic acid attached to the nucleic acid sequencing component. Still another example chemical capture agent includes a member of a binding pair that is capable of binding to a second member of a binding pair that is attached to the nucleic acid sequencing component. Example binding pairs include a NiNTA (nickel-nitrilotriacetic acid) ligand and a histidine tag, or streptavidin or avidin and biotin, etc. Yet another example of the chemical capture agent is a chemical reagent that is capable of forming an electrostatic interaction, a hydrogen bond, or a covalent bond with the nucleic acid sequencing component. Covalent bonds may be formed, for example, through thiol-disulfide exchange, click chemistry, Diels-Alder, Michael additions, amine-aldehyde coupling, amine-acid chloride reactions, amine-carboxylic acid reactions, nucleophilic substitution reactions, etc. Some chemical capture agents may be light-triggered, i.e., activated to chemically bind to the functional agent when exposed to light.

As used herein, “cycloalkyl” refers to a completely saturated (no double or triple bonds) mono- or multi- cyclic hydrocarbon ring system. When composed of two or more rings, the rings may be joined together in a fused fashion. Cycloalkyl groups can contain 3 to 10 atoms in the ring(s). In some examples, cycloalkyl groups can contain 3 to 8 atoms in the ring(s). A cycloalkyl group may be unsubstituted or substituted. Example cycloalkyl groups include cyclopropyl, cyclobutyl, cyclopentyl, cyclohexyl, cycloheptyl, and cyclooctyl.

As used herein, “cycloalkenyl” or “cycloalkene” means a carbocycle ring or ring system having at least one double bond, wherein no ring in the ring system is aromatic. Examples include cyclohexenyl or cyclohexene and norbornenyl or norbornene. Also as used herein, “heterocycloalkenyl” or “heterocycloalkene” means a carbocycle ring or ring system with at least one heteroatom in ring backbone, having at least one double bond, wherein no ring in the ring system is aromatic.

As used herein, “cycloalkynyl” or “cycloalkyne” means a carbocycle ring or ring system having at least one triple bond, wherein no ring in the ring system is aromatic. An example is cyclooctyne. Another example is bicyclononyne. Also as used herein, “heterocycloalkynyl” or “heterocycloalkyne” means a carbocyclyl ring or ring system with at least one heteroatom in ring backbone, having at least one triple bond, wherein no ring in the ring system is aromatic.

The term “depositing,” as used herein, refers to any suitable application technique, which may be manual or automated, and, in some instances, results in modification of the surface properties. Generally, depositing may be performed using vapor deposition techniques, coating techniques, grafting techniques, or the like. Some specific examples include chemical vapor deposition (CVD), spray coating (e.g., ultrasonic spray coating), spin coating, dunk or dip coating, doctor blade coating, puddle dispensing, flow through coating, aerosol printing, screen printing, microcontact printing, inkjet printing, or the like.

As used herein, the term “depression” refers to a discrete concave feature in a substrate having a surface opening that is at least partially surrounded by interstitial region(s) of the substrate.

The term “each,” when used in reference to a collection of items, is intended to identify an individual item in the collection, but does not necessarily refer to every item in the collection. Exceptions can occur if explicit disclosure or context clearly dictates otherwise.

The term “epoxy” (also referred to as a glycidyl or oxirane group) as used herein refers to

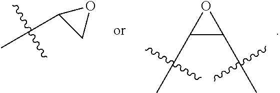

As used herein, the term “electrostatic capture agent” refers to a charged material that is capable of electrostatically anchoring a charged nucleic acid sequencing component or a reversibly chargeable nucleic acid sequencing component. For pre-clustered nucleic acid sequencing components, the attached template nucleic acid strands (amplicons) are negatively charged. As such, positively charged pads, e.g., made of silanes, polymers with azide functional groups, poly-lysine, polyimines (e.g., polyethyleneimine, polypropylene imine, etc.), and other positively charged materials, may be used as the electrostatic capture agent. Another example of an electrostatic capture agent is an electrode that can attract, when a proper voltage is applied, the charged nucleic acid sequencing component. A reversibly chargeable nucleic acid sequencing component can include an amine or a carboxylic acid attached at the polar head (e.g., at the hydrophilic sugar group), which can be reversibly switched between a neutral and a charged species in response to a pH change, and the charged species can be attracted to the electrode. The amine or carboxylic acid would not be attached to the non-polar tail portion, as this would disrupt the bi-layer orientation.

As used herein, the term “flow cell” is intended to mean a vessel having an open or enclosed flow channel where a reaction can be carried out. Some flow cells include an inlet for delivering reagent(s) to the flow channel, and an outlet for removing reagent(s) from the flow channel. In some examples, the flow cell enables the detection of the reaction that occurs in the chamber. For example, the flow cell may include one or more transparent surfaces allowing for the optical detection of arrays, optically labeled molecules, or the like within the flow channel.

As used herein, a “flow channel” or “channel” may be an area defined between two bonded components or an open area defined in one component, which can selectively receive a liquid sample. In some examples, the flow channel may be defined between a substrate and a lid, and thus may be in fluid communication with one or more depressions defined in the substrate or capture sites positioned on the substrate. The flow channel may also be defined between two substrate surfaces that are bonded together. In other examples, the flow channel is defined in an open wafer substrate, which is not bonded to another component. This flow channel is open to the external environment.

A “functional agent” is a material, molecule or moiety that is capable of anchoring to a chemical capture site of a flow cell via a chemical mechanism. One example functional agent includes a target nucleic acid that is complementary to a capture nucleic acid (e.g., a capture oligonucleotide) on the flow cell. Still another example functional agent includes a member of a binding pair that is capable of binding to a second member of a binding pair that is attached to the flow cell.

“Functionalized nanostructures” include a nanostructure core, a glycolipid bi-layer attached to the nanostructure core, primers attached to the glycolipid bi-layer, and a mechanism to attach to a flow cell capture site. The functionalized nanostructures may be used in off flow cell cluster generation.

A “glycolipid bi-layer” is a membrane composed of two layers of glycolipid molecules. Each glycolipid molecule includes a lipid (which includes a hydrophobic (non-polar) lipid tail) and a hydrophilic (polar) sugar group. The glycolipids in a single bi-layer may include the same sugar groups or different sugar groups. In some examples, a hetero-lipid bi-layer may be formed in which some of the molecules are lipid molecules intermingled with the glycolipid molecules.

As used herein, “heteroaryl” refers to an aromatic ring or ring system (i.e., two or more fused rings that share two adjacent atoms) that contain(s) one or more heteroatoms, that is, an element other than carbon, including but not limited to, nitrogen, oxygen and sulfur, in the ring backbone. When the heteroaryl is a ring system, every ring in the system is aromatic. The heteroaryl group may have 5-18 ring members.

As used herein, “heterocycle” means a non-aromatic cyclic ring or ring system containing at least one heteroatom in the ring backbone. Heterocycles may be joined together in a fused, bridged or spiro-connected fashion. Heterocycles may have any degree of saturation provided that at least one ring in the ring system is not aromatic. In the ring system, the heteroatom(s) may be present in either a non-aromatic or aromatic ring. The heterocycle group may have 3 to 20 ring members (i.e., the number of atoms making up the ring backbone, including carbon atoms and heteroatoms). In some examples, the heteroatom(s) are O, N, or S.

The term “hydrazine” or “hydrazinyl” as used herein refers to a —NHNH₂ group.

As used herein, the term “hydrazone” or “hydrazonyl” as used herein refers to a

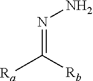

group in which Rₐ and R_{b} are each independently selected from hydrogen, C1-6 alkyl, C2-6 alkenyl, C2-6 alkynyl, C3-7 carbocyclyl, C6-10 aryl, 5-10 membered heteroaryl, and 5-10 membered heterocyclyl, as defined herein.

As used herein, “hydroxy” or “hydroxyl” refers to an —OH group.

The term “interstitial region” refers to an area, e.g., of a substrate that separates depressions or capture sites. For example, an interstitial region can separate one depression of an array from another depression of the array. The two depressions that are separated from each other can be discrete, i.e., lacking physical contact with each other. In many examples, the interstitial region is continuous whereas the depressions are discrete, for example, as is the case for a plurality of depressions defined in an otherwise continuous surface. In other examples, the interstitial regions and the features are discrete, for example, as is the case for a plurality of trenches separated by respective interstitial regions. The separation provided by an interstitial region can be partial or full separation. Interstitial regions may have a surface material that differs from the surface material of the depressions defined in the surface. For example, depressions can have a glycolipid bi-layer and primers, and the interstitial regions can be free of the glycolipid bi-layer and primers.

A “ketone” has the structure R₂C═O, where R can be a variety of carbon-containing substituents, such as C1-6 alkyl C2-6 alkenyl, C2-6 alkynyl, C3-7 carbocyclyl, C6-10 aryl, 5-10 membered heteroaryl, and 5-10 membered heterocyclyl, as defined herein.

As used herein, the term “magnetic capture agent” refers to a magnetic material that is capable of magnetically anchoring a functionalized nanostructure. Example magnetic capture agents include ferromagnetic materials and ferrimagnetic materials.

As used herein, the term “mechanism” refers to a functional agent or a magnetic material that is incorporated into the nucleic acid sequencing component, or to the charged amplicons at the surface of the nucleic acid sequencing component, each of which renders the nucleic acid sequencing component capable of anchoring to a capture site in a flow cell.

“Nitrile oxide,” as used herein, means a “RₐC≡N⁺O⁻” group in which Rₐ is defined herein. Examples of preparing nitrile oxide include in situ generation from aldoximes by treatment with chloramide-T or through action of base on imidoyl chlorides [RC(Cl)═NOH] or from the reaction between hydroxylamine and an aldehyde.

“Nitrone,” as used herein, means a

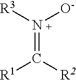

group in which R¹, R², and R³ may be any of the Rₐ and R_{b} groups defined herein, except that R³ is not hydrogen (H).

A “nucleic acid sequencing component” includes a support, a glycolipid bi-layer attached to the support, and primers attached to the glycolipid bi-layer. Each example of the nucleic acid sequencing component can be used for generating a cluster of amplicons (i.e., replicated template nucleic acid strands) that is used sequencing. One example of the nucleic acid sequencing component is a flow cell, and another example of the nucleic acid sequencing component is a functionalized nanostructure.

As used herein, a “nucleotide” includes a nitrogen containing heterocyclic base, a sugar, and one or more phosphate groups. Nucleotides are monomeric units of a nucleic acid sequence. In ribonucleic acids (RNA), the sugar is a ribose, and in deoxyribonucleic acids (DNA), the sugar is a deoxyribose, i.e., a sugar lacking a hydroxyl group that is present at the 2′ position in ribose. The nitrogen containing heterocyclic base (i.e., nucleobase) can be a purine base or a pyrimidine base. Purine bases include adenine (A) and guanine (G), and modified derivatives or analogs thereof. Pyrimidine bases include cytosine (C), thymine (T), and uracil (U), and modified derivatives or analogs thereof. The C-1 atom of deoxyribose is bonded to N-1 of a pyrimidine or N-9 of a purine. A nucleic acid analog may have any of the phosphate backbone, the sugar, or the nucleobase altered. Examples of nucleic acid analogs include, for example, universal bases or phosphate-sugar backbone analogs, such as peptide nucleic acid (PNA).

The terms “polymeric hydrogel” and “hydrogel” refer to a semi-rigid polymer that is permeable to liquids and gases. The polymeric hydrogel can swell when liquid (e.g., water) is taken up and that can contract when liquid is removed, e.g., by drying. While a hydrogel may absorb water, it is not water-soluble.

The term “orthogonal,” when used to describe two cleaving chemistries means that the chemistries are different from each other. Orthogonal cleaving chemistries are susceptible to different cleaving agents so that the first cleaving chemistry is unaffected when exposed to the cleaving agent for the second cleaving chemistry, and the second cleaving chemistry is unaffected when exposed to the cleaving agent for the first cleaving chemistry.

As used herein, the term “polyhedral oligomeric silsesquioxane” (commercially available under the tradename POSS® from Hybrid Plastics) refers to a chemical composition that is a hybrid intermediate (e.g., RSiO_{1.5}) between that of silica (SiO₂) and silicone (R₂SiO). An example of polyhedral oligomeric silsesquioxane can be that described in Kehagias et al., Microelectronic Engineering 86 (2009), pp. 776-778, which is incorporated by reference in its entirety. In an example, the composition is an organosilicon compound with the chemical formula [RSiO_{3/2}]ₙ, where the R groups can be the same or different. Example R groups for polyhedral oligomeric silsesquioxane include epoxy, azide/azido, a thiol, a poly(ethylene glycol), a norbornene, a tetrazine, acrylates, and/or methacrylates, or further, for example, alkyl, aryl, alkoxy, and/or haloalkyl groups. The resin composition disclosed herein may comprise one or more different cage or core structures as monomeric units. The average cage content can be adjusted during the synthesis, and/or controlled by purification methods, and a distribution of cage sizes of the monomeric unit(s) may be used in the examples disclosed herein.

As used herein, the term “primer” is defined as a single stranded nucleic acid sequence (e.g., single strand DNA). Some primers are part of a primer set, which serve as a starting point for template amplification and cluster generation. Other primers, referred to herein as sequencing primers, serve as a starting point for DNA synthesis. The 5′ terminus of each primer in a primer set may be modified to allow a coupling reaction with a functional group of the glycolipid bi-layer. The primer length can be any number of bases long and can include a variety of non-natural nucleotides. In an example, the sequencing primer is a short strand, ranging from 10 to 60 bases, or from 20 to 40 bases.

The term “primer set” refers to a pair of primers that together enable the amplification of a template nucleic acid strand (also referred to herein as a library template). Opposed ends of the template strand include adapters to hybridize to the respective primers in a set.

The term “support” refers to a structure upon which the glycolipid bi-layer is added. The support may be a substrate or a core nanostructure. The “substrate” refers to a substantially planar material or stack of materials that can be patterned with a lane or depressions for confining at least the glycolipid bi-layer and primers. The “core nanostructure” refers to any nano-sized structure, including spherical nanoparticles or non-spherical nanoparticles, such as cubes, triangular prisms, rod shaped, platelets, cage-like (e.g., non-spherical, hollow particles having a porous shell), tubes, etc. that can be functionalized with at least the glycolipid bi-layer and primers. Examples of suitable supports will be described further herein.

A “thiol” functional group refers to —SH.

As used herein, the terms “tetrazine” and “tetrazinyl” refer to six-membered heteroaryl group comprising four nitrogen atoms. Tetrazine can be optionally substituted.

“Tetrazole,” as used herein, refers to five-membered heterocyclic group including four nitrogen atoms. Tetrazole can be optionally substituted.

The term “transparent” refers to a material, e.g., in the form of a layer, that is capable of transmitting a particular wavelength or range of wavelengths. For example, the material may be transparent to wavelength(s) that are used in a sequencing operation. Transparency may be quantified using transmittance, i.e., the ratio of light energy falling on a body to that transmitted through the body. The transmittance of a transparent layer will depend upon the thickness of the layer, the wavelength of light, and the dosage of the light to which it is exposed. In the examples disclosed herein, the transmittance of the transparent metal layer may range from 0.1 (10%) to 1 (100%). The material of the transparent metal layer may be a pure material, a material with some impurities, or a mixture of materials, as long as the resulting layer is capable of the desired transmittance.

### Nucleic Acid Sequencing Components

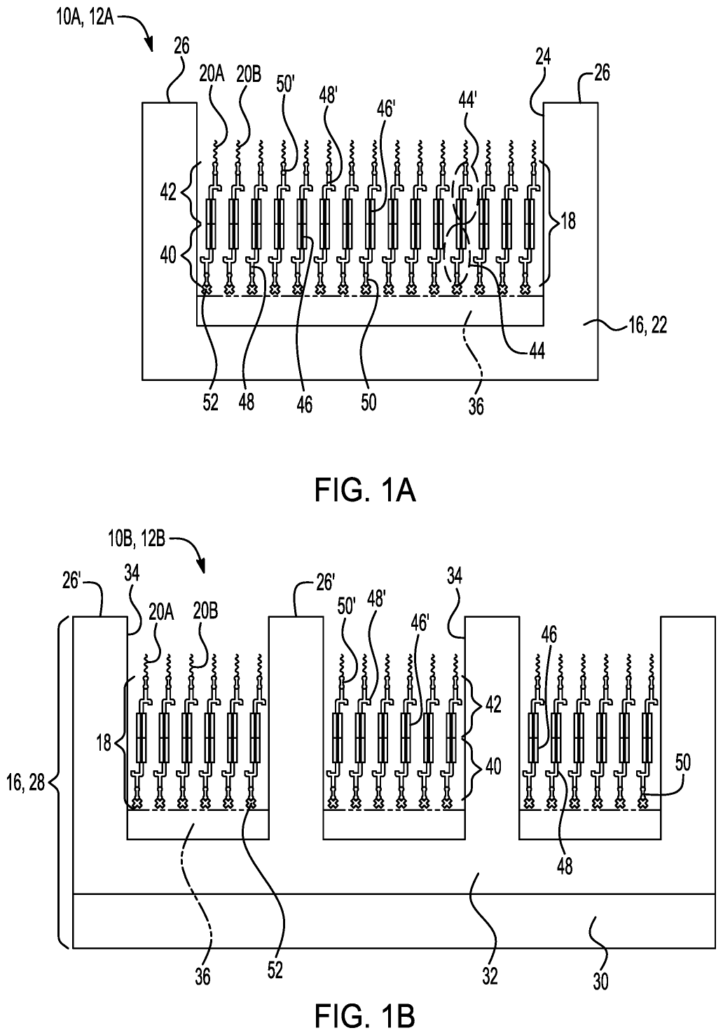

Different examples of the nucleic acid sequencing component 10A, 10B, 10C are shown in FIG. 1A, FIG. 1B, and FIG. 1C. In the examples shown in FIG. 1A and FIG. 1B, the nucleic acid sequencing component 10A, 10B is a flow cell 12A, 12B. In the example shown in FIG. 1C, the nucleic acid sequencing component 10C is a functionalized nanostructure 14. Each example of the nucleic acid sequencing component 10A, 10B, 10C disclosed herein includes a support 16; a glycolipid bi-layer 18 attached to at least a portion of the support 16; and first and second primers 20A, 20B respectively attached to the glycolipid bi-layer 18.

In the example shown in FIG. 1A, the support 16 is a single layered substrate 22 that includes a lane 24 surrounded by interstitial (and in this example bonding) regions 26; and the glycolipid bi-layer 18 and the first and second primers 20A, 20B are positioned within the lane 24. In the example shown in FIG. 1B, the support 16 is a multi-layer substrate 28 that includes depressions 34 surrounded by interstitial regions 26′; and the glycolipid bi-layer 18 and the first and second primers 20A, 20B are positioned within at least some of the depressions 34. In the example shown in FIG. 1C, the support 16 is a core nanostructure 38 and the glycolipid bi-layer 18 and the first and second primers 20A, 20B are positioned at the surface of the core nanostructure 38.

### Support

The nucleic acid sequencing component 10A in FIG. 1A is one example of the flow cell 12A. In this example, the support 16 is a single layer substrate 22 that has a lane 24 defined therein.

Examples of suitable single layer substrates 22 include epoxy siloxane, glass, modified or functionalized glass, plastics (including acrylics, polystyrene and copolymers of styrene and other monomers, polypropylene, polyethylene, polybutylene, polyurethanes, polytetrafluoroethylene (such as TEFLON® from Chemours), cyclic olefins/cyclo-olefin polymers (COP) (such as ZEONOR® from Zeon), polyimides, etc.), nylon (polyamides), ceramics/ceramic oxides, silica, fused silica, or silica-based materials, aluminum silicate, silicon and modified silicon (e.g., boron doped p+ silicon), silicon nitride (Si₃N₄), silicon oxide (SiO₂), tantalum pentoxide (Ta₂O₅) or other tantalum oxide(s) (TaOₓ), hafnium oxide (HfO₂), carbon, metals, or the like.

The single layer substrate 22 may be a circular sheet, panel, wafer, die, etc. having a diameter ranging from about 2 mm to about 300 mm, e.g., from about 200 mm to about 300 mm, or may be a rectangular sheet, panel, wafer, die etc. having its largest dimension up to about 10 feet (˜3 meters). Sheets, panels, and wafers may subsequently be diced to form an individual substrate 22. In one example, the substrate 22 is a die having a width ranging from about 0.1 mm to about 10 mm. While example dimensions have been provided, it is to be understood that single layer substrate 22 with any suitable dimensions may be used.

The lane 24 is a concave region that is surrounded by interstitial regions 26 of the single layer substrate 22. The interstitial regions 26 completely surround the lane 24, and may provide a bonding surface where a lid or a second single layer substrate 22 may be attached to form an enclosed flow cell. While a single lane 24 is shown in FIG. 1A, it is to be understood that multiple lanes 24 may be defined in the single layer substrate 22, where each of the lanes 24 is fluidically separate in the final flow cell 12A so that each lane 24 provides a discrete reaction vessel.

The dimensions, shape, and depth of the lane 24 will depend, in part, upon the dimensions, shape, and thickness of the single layer substrate 22. In one example, the lane 24 shape mimics the shape of the single layer substrate, but has dimensions that are smaller than the single layer substrate 22 so that the depth of the lane 24 is less than the thickness of the substrate 22, and so that interstitial regions 26 of the substrate 22 completely surround the lane 24. In an example, the lane 24 is rectangular.

The nucleic acid sequencing component 10B in FIG. 1B is another example of the flow cell 12B. In this example, the support 16 is a multi-layered substrate 28 that includes a base 30 and a patterned layer 32 over the base 30. The base 30 may be any of the examples set forth herein for the single layer substrate 22. The patterned layer 32 may be any material that is capable of being patterned with depressions 34.

In an example, the patterned layer 32 is an inorganic oxide that is selectively applied to the base 30 in the desired pattern. Examples of suitable inorganic oxides include tantalum oxide (e.g., Ta₂O₅), aluminum oxide (e.g., Al₂O₃), silicon oxide (e.g., SiO₂), hafnium oxide (e.g., HfO₂), etc.

In another example, the patterned layer 32 is a resin that is applied to the base 30 and then patterned. Some examples of suitable resins include a polyhedral oligomeric silsesquioxane-based resin, a non-polyhedral oligomeric silsesquioxane epoxy resin, a poly(ethylene glycol) resin, a polyether resin (e.g., ring opened epoxies), an acrylic resin, an acrylate resin, a methacrylate resin, an amorphous fluoropolymer resin (e.g., CYTOP® from Bellex), and combinations thereof.

The multi-layered substrate 28 may have any of the dimensions set forth herein for the single layer substrate 22.

Each depression 34 is a three-dimensional structure that extends inward (downward) from an adjacent surface. The depression 34 is thus a concave region with respect to the interstitial regions 26′ that surround the depressions 34. Each depression 34 is formed in the patterned layer 32 via etching, photolithography, imprinting, etc. so that the interstitial regions 26′ extend above and completely surround the depression 34.

The depressions 34 may be formed in an array across the patterned layer 32. In some examples, all of the depressions 34 defined in the patterned layer 32 are in fluid communication with a single flow channel of the flow cell 12B. In these examples, interstitial regions 26′ at the perimeter of the patterned layer 32 provide a bonding surface where a lid or a second multi-layered substrate 28 may be attached to form an enclosed flow cell having the single flow channel. In other examples, the flow cell 12B includes several discrete flow channels (e.g., two, four, eight, etc.), and a respective subset of the depressions 34 is in fluid communication with each of the flow channels. In these examples, interstitial regions 26′ that extend the length and width of the patterned layer 32 provide bonding surfaces where the lid or the second multi-layered substrate 28 may be attached to form an enclosed flow cell having multiple flow channels.

Whether arranged to be in fluid communication with a single or multiple flow channels, many different patterns for the depressions 34 are envisaged, including regular, repeating, and non-regular patterns. In an example, the depressions 34 are disposed in a hexagonal grid for close packing and improved density. Other layouts may include, for example, rectangular layouts, triangular layouts, striped layouts, diagonal layouts, etc. In some examples, the layout or pattern can be an x-y format of depressions 34 that are in rows and columns. In still other examples, the layout or pattern can be a random arrangement of depressions 34.

The layout or pattern of the depressions 34 may be characterized with respect to the density of the depressions 34 (e.g., number of depressions 34) in a defined area. For example, the depressions 34 may be present at a density of approximately 2 million per mm². The density may be tuned to different densities including, for example, a density of about 100 per mm², about 1,000 per mm², about 0.1 million per mm², about 1 million per mm², about 2 million per mm², about 5 million per mm², about 10 million per mm², about 50 million per mm², or more, or less. It is to be further understood that the density of depressions 34 can be between one of the lower values and one of the upper values selected from the ranges above. As examples, a high density array may be characterized as having depressions 34 separated by less than about 100 nm, a medium density array may be characterized as having depressions 34 separated by about 400 nm to about 1 μm, and a low density array may be characterized as having depressions 34 separated by greater than about 1 μm. While example densities have been provided, it is to be understood that any suitable densities may be used. In some instances, it may be desirable for the spacing between depressions 34 to be even greater than the examples listed herein.

The layout or pattern of the depressions 34 may also or alternatively be characterized in terms of the average pitch, or the spacing from the center of one depressions 34 to the center of an adjacent depressions 34 (center-to-center spacing) or from the left edge of one depressions 34 to the right edge of an adjacent depressions 34 (edge-to-edge spacing). The pattern can be regular, such that the coefficient of variation around the average pitch is small, or the pattern can be non-regular in which case the coefficient of variation can be relatively large. In either case, the average pitch can be, for example, about 50 nm, about 0.1 μm, about 0.5 μm, about 1 μm, about 5 μm, about 10 μm, about 100 μm, or more or less. The average pitch for a particular pattern of depressions 34 can be between one of the lower values and one of the upper values selected from the ranges above. In an example, the depressions 34 have a pitch (center-to-center spacing) of about 1.5 μm. While example average pitch values have been provided, it is to be understood that other average pitch values may be used.

While any suitable three-dimensional geometry may be used for the depressions 34, a geometry with an at least substantially flat bottom surface may be desirable so that the glycolipid bi-layer 18 or polymeric hydrogel 36 may be formed thereon. Example depression geometries include a sphere, a cylinder, a cube, polygonal prisms (e.g., rectangular prisms, hexagonal prisms, etc.), or the like.

Depressions 34 can have any of a variety of shapes at their opening in the patterned layer 32 including, as examples, round, elliptical, square, polygonal, star shaped (with any number of vertices), etc. The cross-section of a depression taken orthogonally with the surface of the patterned layer 32 can be curved, square, polygonal, hyperbolic, conical, angular, etc.

The size of each depression 34 may be characterized by its volume, opening area, depth, and/or diameter or length and width.

Each depression 34 can have any volume that is capable of receiving the glycolipid bi-layer 18, or the bi-layer 18 and the polymeric hydrogel 36. For example, the volume can be at least about 1×10^{-3 μ}m³, at least about 1×10^{-2 μ}m³, at least about 0.1 μm³, at least about 1 μm³, at least about 10 μm³, at least about 100 μm³, or more. Alternatively or additionally, the volume can be at most about 1×10^{4 μ}m³, at most about 1×10^{3 μ}m³, at most about 100 μm³, at most about 10 μm³, at most about 1 μm³, at most about 0.1 μm³, or less.

The area for each depression opening can be at least about 1×10^{-3 μ}m², at least about 1×10^{-2 μ}m², at least about 0.1 μm², at least about 1 μm², at least about 10 μm², at least about 100 μm², or more. Alternatively or additionally, the area can be at most about 1×10^{3 μ}m², at most about 100 μm², at most about 10 μm², at most about 1 μm², at most about 0.1 μm², at most about 1×10^{-2 μ}m², or less. The area occupied by each depression opening can be greater than, less than or between the values specified above.

The depth of each depression 34 is large enough to house at least the glycolipid bi-layer 18 and the primers 20A, 20B. In an example, the depth may be at least about 0.1 μm, at least about 0.5 μm, at least about 1 μm, at least about 10 μm, at least about 100 μm, or more. Alternatively or additionally, the depth can be at most about 1×10^{3 μ}m, at most about 100 μm, at most about 10 μm, or less. In some examples, the depth is about 0.4 μm. The depth of each depression 34 can be greater than, less than or between the values specified above.

In some instances, the diameter or each of the length and width of each depression 34 can be at least about 50 nm, at least about 0.1 μm, at least about 0.5 μm, at least about 1 μm, at least about 10 μm, at least about 100 μm, or more. Alternatively or additionally, the diameter or each of the length and width can be at most about 1×10^{3 μ}m, at most about 100 μm, at most about 10 μm, at most about 1 μm, at most about 0.5 μm, at most about 0.1 μm, or less (e.g., about 50 nm). In some examples, the diameter or each of the length and width is about 0.4 μm. The diameter or each of the length and width of each depression 34 can be greater than, less than or between the values specified above.

Referring now to FIG. 1C, another of the nucleic acid sequencing components 10C is depicted. In this example, the nucleic acid sequencing component 10C is a functionalized nanostructure 14, and the support 16 is a core nanostructure 38.

The core nanostructure 38 may be a spherical nanoparticle, or a non-spherical nanoparticle, such as a cube, a triangular prism, rod shaped, a platelet, cage-like (e.g., non-spherical, hollow particles having a porous shell), a tube, etc. In still example, the core nanostructure 38 may be an irregularly shaped nanoparticle.

The dimensions of the core nanostructure 38 may vary depending upon its shape. In the examples disclosed herein, the largest dimension (e.g., diameter, length, median, etc.) of the core nanostructure 38 is on the nanoscale, and thus ranges from about 1 nm to less than 1000 nm. In some examples, the core nanostructure 38 are nanoparticles having a diameter of greater than or equal to 1 nm, 2 nm, 3 nm, 4 nm, 5 nm, 6 nm, 7 nm, 8 nm, 9 nm, 10 nm, 20 nm, 30 nm, 40 nm, 50 nm, 60 nm, 70 nm, 80 nm, 90 nm, or greater than or equal to 100 nm.

Examples of suitable materials for the core nanostructure 38 include magnetic materials (e.g., magnetic FeOₓ, silica coated FeOₓ), polymers (e.g., polytetrafluoroethylene (PTFE), some polyacrylics, polypropylene, polyethylene, polybutylene, polyurethanes, polystyrene or styrene copolymers, nylon (i.e., polyamide), and polycaprolactone (PCL)), nitrocellulose, silica (SiO₂), silica-based materials (e.g., functionalized SiO₂), carbon, metals (e.g., gold, silver, tin, rhodium, ruthenium, palladium, osmium, iridium, platinum, copper, aluminum, etc.), doped semi-metals (e.g., doped silicon), direct bandgap semiconductors (e.g., gallium arsenide), metal composites, metal alloys, and any of the polymeric hydrogels described herein in reference to reference numeral 36. Some other example materials for the core nanostructure 38 include PEG-acrylate, PEG-diacrylate, PEG-amine, PEG-carboxylate, PEG-dithiol, PEG-epoxide, PEG-isocyanate, PEG-maleimide, crosslinked poly(methyl methacrylate) (PMMA), polyvinylpyrrolidone (PVPON), polyvinyl alcohol (PVA), polyethylene oxide-polypropylene oxide block copolymers (PEO-PPO), poly(hydroxyethyl methacrylate) (PHEMA), poly(N-isopropylacrylamide) (PNIPAAm), poly(lactic acid)-poly(ethylene glycol) block copolymers, poly(ethylene glycol)-poly(lactic-co-glycolic acid) block copolymers, poly(acrylic-co-vinylsulfonic acid), poly(acrylamide-co-vinylsulfonic acid), poly(L-aspartic acid), poly(aspartamide), adipic dihydrazide modified or aldehyde modified poly(L-glutamic acid), bisacrylamide, or hydrogels based on one or more of polylysine, starch, agar, agarose, heparin, alginate, alginate sulfate, dextran sulfate, hyaluronan, pectin, carrageenan, gelatin, chitosan, cellulose, and collagen, or combinations or mixtures thereof.

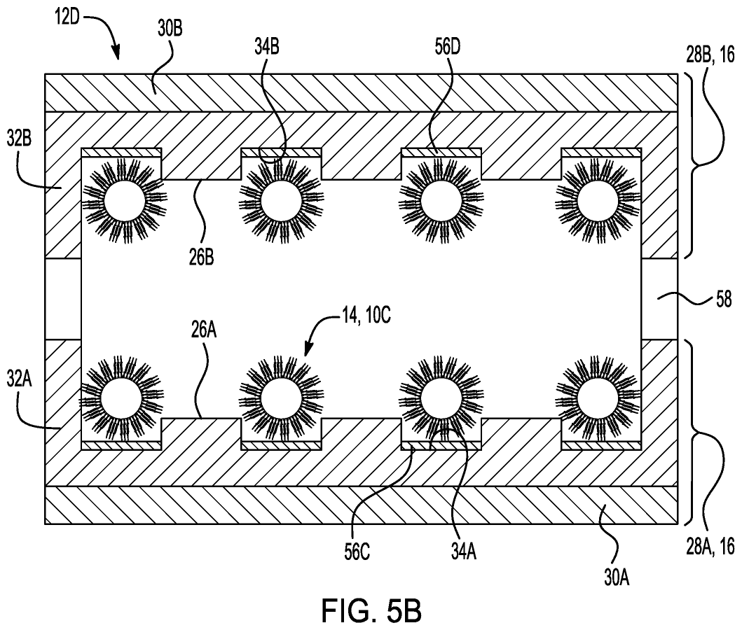

The core nanostructure 38 may each have a solid structure, a hollow structure, or a core-shell structure. The core-shell structure has one of the listed materials at the interior and another of the listed materials at the exterior, which at least partially encapsulates the interior. In an example, a magnetic material (e.g., nickel, iron, cobalt, or other ferromagnetic materials, ferrites, magnetite, or other ferromagnetic materials, etc.) may be incorporated into the interior or into the exterior of the core-shell structure. As one example, the magnetic material may be used as the interior and a metal or polymer may be used as the exterior. This example core-shell structure may be suitable for use when the flow cell (described below in reference to FIG. 5A through FIG. 5C) includes a magnetic capture agent, because the magnetic material is the mechanism for attachment to the flow cell capture site.

### Glycolipid Bi-Layer

Each example of the nucleic acid sequencing component 10A, 10B, 10C includes the glycolipid bi-layer 18 attached to at least a portion of the support 16, 22 or 16, 28, or 16, 38.

Each example of the glycolipid bi-layer 18 includes a first portion 40 including first glycolipid residues 44 attached to the portion of the support 16, 22 or 16, 28, or 16, 38, wherein each of the first glycolipid residues 44 includes a first hydrophobic tail 46 and a first hydrophilic sugar group 48; and a second portion 42 including second glycolipid residues 44′ self-assembled with the first lipid residues 44, wherein each of the second glycolipid residues 44′ includes a second hydrophobic tail 46′ and a second hydrophilic sugar group 48′.

The hydrophobic tails 46, 46′ are part of respective lipids, which are independently selected from the group consisting of a bacterial phospholipid and an archaeal lipid. Specific examples of bacterial phospholipids include those in the following families: phosphatidylehanolamine, phosphatidylglycerol, cardiolipin, phosphatidylcholine, and phosphatidylinositol. Other examples of suitable lipids (containing the hydrophobic tails 46, 46′) include phosphorus-free glyercolipids, serineglycine-containing lipids, sphingolipids, hopanoids, ornithine lipids, and sulfonolipids. Specific example of archaeal lipids include diether lipids: archaeol or caldarchaeol, C25 Sn-2,3 sesterterpanyl glycerol, C20 macrocyclic archaeol, C20 phytanyl-C25 sesterterpanly glycerol, tetriol-diphytanyl diether, isocaldarchaeol, caldarchaeol H-shaped, nonitol-tetraether derivate, and crenarchaeol/tetraethers with cyclopentane rings.

The sugar groups 48, 48′ are independently selected from the group consisting of a six-carbon sugar, a five-carbon sugar, a four-carbon sugar, a seven-carbon sugar, a deoxysugar, a di-deoxysugar, an acidic sugar, a sugar alcohol, and combinations thereof.

Examples of six-carbon sugars include hexose, D-glucose, D-galactofuranose, D-galactose, L-galactose, D-mannose, D-allose, L-altrose, D-gulose, L-idose, and D-talose. Derivatives of six-carbon sugars that may be used include amino sugars, some of which are hexose with an amino group at the 2-position (e.g., D-glucosamine, D-galactosamine, D-mannosamine, D-allosamine, L-altrosamine, D-gulosamine, L-idosamine, and D-talosamine) and others of which are hexose with an N-acetylated amino group at the 2-position (e.g., N-acetyl-D-glucosamine, N-acetyl-D-galactosamine, N-acetyl-D-mannosamine, N-acetyl-D-allosamine, N-acetyl-L-altrosamine, N-acetyl-D-gulosamine, N-acetyl-L-idosamine, N-acetyl-D-talosamine).

Examples of five-carbon sugars include D-ribose, D-arabinose, L-arabinose, D-xylose, and S-lyxose.

Examples of four-carbon sugars include D-erythrose and D-threose.

Examples of seven-carbon sugars include heptose, L-glycero-D-manno-heptose, and D-glycero-D-manno-heptose.

Deoxysugars are derivatives of hexose or pentose without a hydroxyl group at the 6-position (in hexose) or the 2-position (in pentose). Some examples of deoxysugars include 6-deoxy-L-altrose, 6-deoxy-D-gulose, 6-deoxy-D-talose, D-fucose, L-fucose, D-rhamnose, L-rhamnose, D-quinovose, 2-deoxyglucose, and 2-deoxyribose. Some deoxysugars are also amino sugars, such as N-acetyl-D-fucosamine, N-acetyl-L-fucosamine, N-acetyl-L-rhamnosamine, N-acetyl-D-quinovosamine, N-acetyl-6-deoxy-L-altrosamine, and N-acetyl-6-deoxy-D-talosamine.

Di-deoxysugars are derivatives of hexose without hydroxyl groups at the 6-position and at another position. Some examples of di-deoxysugars include olivose, tyvelose, ascarylose, abequose, paratose, digitoxose, and colitose.

Acidic sugars are sugar derivatives that include an acid functional group, e.g., within the sugar ring, appended to the sugar ring, attached to the sugar chain, etc. Some example acidic sugars include uronic acid sugars, which are hexose derivatives having a negatively charged carboxylate at the 6-position. Examples of uronic acid sugars include D-glucuronic acid, D-galacturonic acid, D-mannuronic acid, D-alluronic acid, L-altruronic acid, D-guluronic acid, L-guluronic acid, L-iduronic acid, and D-taluronic acid. Other example acidic sugars include sialic acid sugars, which are nine carbon acidic sugars. Examples of sialic acid sugars include sialic acid, neuraminic acid, N-actylneuraminic acid, and N-glycolylneuraminic acid.

Suitable sugar alcohols include erythritol, arabinitol, xylitol, ribitol, glucitol, galactitol, and mannitol.

Other suitable sugar groups include D-psicose, D-fructose, L-sorbose, D-tagatose, D-xylulose, D-sedoheptulose, apiose, bacillosamine, thevetose, acofriose, L-acofriose, cymarose, muramic acid, N-actylmuramic acid, N-glycolylmuramic acid, 3-deoxy-lyxo-heptulosaric acid, ketodeoxyoctonic acid, ketodeoxynononic acid, pseudaminic acid, acinetaminic acid, legionaminic acid, and 4-epilegionaminic acid.

It is to be understood that the glycolipid residues 44, 44′ in the respective portions 40, 42 may be the same (i.e., have the same hydrophobic tail 46, 46′ and the same hydrophilic sugar group 48, 48′) or may be different (i.e., the hydrophobic tails 46, 46′ and/or the hydrophilic sugar groups 48, 48′ are different). Moreover, different glycolipid residues 44 or 44′ may be present within each portion 40 or 42. For example, all of glycolipid residues 44 in the portion 40 may have the same type of hydrophobic tail 46, but some of the glycolipid residues 44 have one type of sugar group 48 while the other glycolipid residues 44 have a different type of sugar group 48. For another example, all of glycolipid residues 44′ in the portion 42 may have the same type of sugar group 48′, but some of the glycolipid residues 44′ have one type of hydrophobic tail 46′ while the other glycolipid residues 44′ have a different type of hydrophobic tail 46′.

Each of the first glycolipid residues 44 also includes a functional group 50 that is intrinsic of, or is attached to, the sugar group 48. In some instances, the functional group 50 is an intrinsic chemical group of the sugar group 48 that is capable of attaching to a group 52 located at the surface of the support 16, 22 or 16, 28, or 16, 38 or of the polymeric hydrogel 36 (when it is included). As examples, D-amino glucose, the monomer of chitosan, presents a primary amine; D-mannuronic acid and L-guluronic acid, the monomers of alginate, present carboxylic acids; and glucuronic acid, a monomer of hyaluronic acid, also presents a carboxylic acid group. Alternatively, the hydroxyl group(s) intrinsically found in several sugar groups 48 can be chemically derivatized with the functional group 50. Examples of such functional groups 50 include an azide, an allyl, a carboxylic acid, an amine, thiol, an aldehyde, and a catechol, each of which can, in turn, be attached to the group 52 located at the surface of the support 16, 22 or 16, 28, or 16, 38 or of the polymeric hydrogel 36 (when it is included). The group 52 at the surface of the support 16, 22 or 16, 28, or 16, 38 may be a functional group of the support material or of an additional layer (e.g., a silane) applied over the support material. One specific example of the functional group 50 is an azide, which can attach to an alkyne or norbornene (e.g., group 52) of a silane that is positioned over the support 16, 22 or 16, 28, or 16, 38 or to an alkyne (e.g., group 52) of the polymeric hydrogel 36.

Each of the second glycolipid residues 44′ also includes a functional group 50′ that is intrinsic of, or is attached to, the sugar group 48′. In some instances, the functional group 50′ is an intrinsic chemical group of the sugar group 48′, such as an amine or a carboxylic acid, which is capable of attaching to a biomolecule, such as an oligonucleotide, via a bio-conjugation reaction. Alternatively, the hydroxyl group(s) intrinsically found in several sugar groups 48′ can be chemically derivatized with the functional group(s) 50′. Examples of such functional groups 50′ include an azide, a carboxylic acid, an amine, thiol, an aldehyde, an alkyne, or a ketone, each of which can, in turn, be attached to a terminal group of the primers 20A, 20B. As examples, a hydroxyl group of the sugar group 48′ may have an azide attached via an imidazolium linker or an ethylene oxide linker.

The following are specific examples of the attachment between the functional group 50′ and the primers 20A, 20B: an amine functional group 50′ may be reacted with a succinimidyl (NHS) ester terminated primer 20A, 20B, or a hydrazine functional group 50′ may be reacted with an aldehyde terminated primer 20A, 20B, an azide functional group 50′ may be reacted with an alkyne terminated primer 20A, 20B, or an alkyne or DBCO (dibenzocyclooctyne) functional group 50′ may be reacted with an azide terminated primer 20A, 20B, or an activated carboxylate group or NHS ester functional group 50′ may be reacted with an amino terminated primer 20A, 20B.

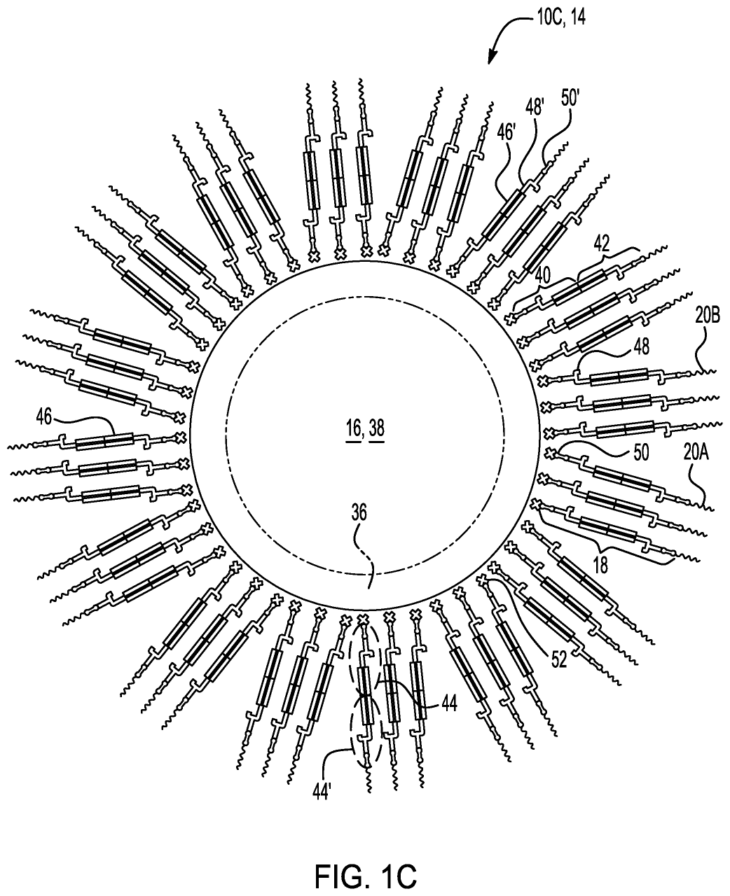

Examples of methods for forming the glycolipid bi-layer 18 are described below in reference to FIG. 2 through FIG. 4.

### Polymeric Hydrogel

Some examples of the nucleic acid sequencing component 10A, 10B, 10C include a polymeric hydrogel 36 (shown in phantom) applied in the lane 24 (FIG. 1A), in the depressions 34 (FIG. 1B), or over the surface of the core nanostructure 38 (FIG. 1C). In these instances, the polymeric hydrogel 36 attaches to the underlying surface (e.g., the support 16) and also includes surface groups to attach the glycolipid bi-layer 18.

In the example shown in FIG. 1A and in FIG. 1B, the support 16 is the substrate 22 or 28, and the nucleic acid sequencing component 10A or 10B further comprises the hydrogel layer 36 attached to at least a portion (e.g., in the lane 24 or in the depressions 34) of the substrate 22 or 28; and the glycolipid bi-layer 18 is attached to the hydrogel layer 36. In the example shown in FIG. 1C, the support 16 is the core nanostructure 38, and the nucleic acid sequencing component 10C further comprises the hydrogel layer 36 attached to core nanostructure 38, wherein the glycolipid bi-layer 18 is attached to the hydrogel layer 36.

The polymeric hydrogel 36 may be any gel material that can swell when liquid is taken up and can contract when liquid is removed, e.g., by drying. In an example, the polymeric hydrogel 36 includes an acrylamide copolymer. Some examples of the acrylamide copolymer are represented by the following structure (I):

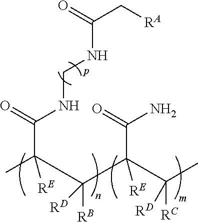

wherein:
R^{A} is selected from the group consisting of azido, optionally substituted amino, optionally substituted alkenyl, optionally substituted alkyne, halogen, optionally substituted hydrazone, optionally substituted hydrazine, carboxyl, hydroxy, optionally substituted tetrazole, optionally substituted tetrazine, nitrile oxide, nitrone, sulfate, and thiol;
R^{B} is H or optionally substituted alkyl;
R^{C}, R^{D}, and R^{E} are each independently selected from the group consisting of H and optionally substituted alkyl;
each of the —(CH₂)ₚ— can be optionally substituted;
p is an integer in the range of 1 to 50;
n is an integer in the range of 1 to 50,000; and
m is an integer in the range of 1 to 100,000.

It is to be understood that some of the R^{A} groups may attach the polymeric hydrogel 36 to the support 16. Some other of the R^{A} groups are the surface groups 52, which can attach to the glycolipid bi-layer 18.

One specific example of the acrylamide copolymer represented by structure (I) is poly(N-(5-azidoacetamidylpentyl)acrylamide-co-acrylamide, PAZAM.

One of ordinary skill in the art will recognize that the arrangement of the recurring “n” and “m” features in structure (I) are representative, and the monomeric subunits may be present in any order in the polymer structure (e.g., random, block, patterned, or a combination thereof).

The molecular weight of the acrylamide copolymer may range from about 5 kDa to about 1500 kDa or from about 10 kDa to about 1000 kDa, or may be, in a specific example, about 312 kDa.

In some examples, the acrylamide copolymer is a linear polymer. In some other examples, the acrylamide copolymer is a lightly cross-linked polymer.

In other examples, the gel material may be a variation of structure (I). In one example, the acrylamide unit may be replaced with N,N-dimethylacrylamide

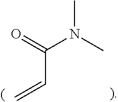

In this example, the acrylamide unit in structure (I) may be replaced with,

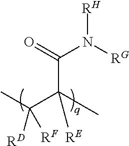

where R^{D}, R^{E}, and R^{F} are each H or a C1-C6 alkyl, and R^{G} and R^{H} are each a C1-C6 alkyl (instead of H as is the case with the acrylamide). In this example, q may be an integer in the range of 1 to 100,000. In another example, the N,N-dimethylacrylamide may be used in addition to the acrylamide unit. In this example, structure (I) may include

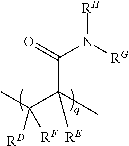

in addition to the recurring “n” and “m” features, where R^{D}, R^{E}, and R^{F} are each H or a C1-C6 alkyl, and R^{G} and R^{H} are each a C1-C6 alkyl. In this example, q may be an integer in the range of 1 to 100,000.

As another example of the polymeric hydrogel 22, the recurring “n” feature in structure (I) may be replaced with a monomer including a heterocyclic azido group having structure (II):

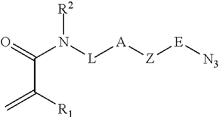

wherein R₁ is H or a C1-C6 alkyl; R² is H or a C1-C6 alkyl; L is a linker including a linear chain with 2 to 20 atoms selected from the group consisting of carbon, oxygen, and nitrogen and 10 optional substituents on the carbon and any nitrogen atoms in the chain; E is a linear chain including 1 to 4 atoms selected from the group consisting of carbon, oxygen and nitrogen, and optional substituents on the carbon and any nitrogen atoms in the chain; A is an N substituted amide with an H or a C1-C4 alkyl attached to the N; and Z is a nitrogen containing heterocycle. Examples of Z include 5 to 10 carbon-containing ring members present as a single cyclic structure or a fused structure. Some specific examples of Z include pyrrolidinyl, pyridinyl, or pyrimidinyl.

As still another example, the gel material may include a recurring unit of each of structure (Ill) and (IV):

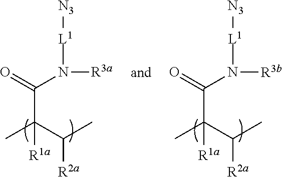

wherein each of R^{1a}, R^{2a}, R^{1b} and R^{2b} is independently selected from hydrogen, an optionally substituted alkyl or optionally substituted phenyl; each of R^{3a} and R^{3b} is independently selected from hydrogen, an optionally substituted alkyl, an optionally substituted phenyl, or an optionally substituted C7-C14 aralkyl; and each L¹ and L² is independently selected from an optionally substituted alkylene linker or an optionally substituted heteroalkylene linker.

In still another example, the acrylamide copolymer is formed using nitroxide mediated polymerization, and thus at least some of the copolymer chains have an alkoxyamine end group. In the copolymer chain, the term “alkoxyamine end group” refers to the dormant species —ONR₁R₂, where each of R₁ and R₂ may be the same or different, and may independently be a linear or branched alkyl, or a ring structure, and where the oxygen atom is attached to the rest of the copolymer chain. In some examples, the alkoxyamine may also be introduced into some of the recurring acrylamide monomers, e.g., at position R^{A} in structure (I). As such, in one example, structure (I) includes an alkoxyamine end group; and in another example, structure (I) includes an alkoxyamine end group and alkoxyamine groups in at least some of the side chains.

It is to be understood that other molecules may be used to form the polymeric hydrogel 36, as long as they are capable of attaching to the support 16 and to the functional groups 50 of the residues 44. Some examples of suitable materials for the polymeric hydrogel 36 include functionalized silanes, such as norbornene silane, azido silane, alkyne functionalized silane, amine functionalized silane, maleimide silane, or any other silane having functional groups that can respectively to the support 16 and to the functional groups 50. Still other examples of suitable materials for polymeric hydrogel 36 include those having a colloidal structure, such as agarose; or a polymer mesh structure, such as gelatin; or a cross-linked polymer structure, such as polyacrylamide polymers and copolymers, silane free acrylamide (SFA), or an azidolyzed version of SFA. Examples of suitable polyacrylamide polymers may be synthesized from acrylamide and an acrylic acid or an acrylic acid containing a vinyl group, or from monomers that form [2+2] photo-cycloaddition reactions. Still other examples of suitable materials for the polymeric hydrogel 36 include mixed copolymers of acrylamides and acrylates. A variety of polymer architectures containing acrylic monomers (e.g., acrylamides, acrylates etc.) may be utilized in the examples disclosed herein, such as branched polymers, including dendrimers (e.g., multi-arm or star polymers), star-shaped or star-block polymers, and the like. For example, the monomers (e.g., acrylamide, acrylamide containing the catalyst, etc.) may be incorporated, either randomly or in block, into the branches (arms) of a dendrimer.

The gel material for the polymeric hydrogel 36 may be formed using any suitable copolymerization process, such as nitroxide mediated polymerization (NMP), reversible addition-fragmentation chain-transfer (RAFT) polymerization, etc.

### Primers

Each example of the nucleic acid sequencing component 10A, 10B, 10C includes the primers 20A, 20B attached to the glycolipid bi-layer 18. The primers 20A, 20B are two different primers of a primer set that are used in sequential paired end sequencing. In sequential paired end sequencing, the primer set is used to amplify a nucleic acid template that has seeded to one of the two primers 20A, 20B. In an example, forward strands are generated, sequenced and removed, and then reverse strands are generated, sequenced, and removed.

As examples, the primer set may include P5 and P7 primers, P15 and P7 primers, or any combination of the PA primers, the PB primers, the PC primers, and the PD primers set forth herein. As example combinations, the primer set may include any two PA, PB, PC, and PD primers, or any combination of one PA primer and one PB, PC, or PD primer, or any combination of one PB primer and one PC or PD primer, or any combination of one PC primer and one PD primer.

Examples of P5 and P7 primers are used on the surface of commercial flow cells sold by Illumina Inc. for sequencing, for example, on HISEQ™, HISEQX™, MISEQ™, MISEQDX™, MINISEQ™, NEXTSEQ™, NEXTSEQDX™, NOVASEQ™, ISEQ™, GENOME ANALYZER™, and other instrument platforms.

The P5 primer is:

P5: 5′ → 3′
(SEQ. ID. NO. 1)
AATGATACGGCGACCACCGAGAUCTACAC

The P7 primer may be any of the following:

P7 #1: 5′ → 3′
(SEQ. ID. NO. 2)
CAAGCAGAAGACGGCATACGAnAT
P7 #2: 5′ → 3′
(SEQ. ID. NO. 3)
CAAGCAGAAGACGGCATACnAGAT
P7 #3: 5′ → 3′
(SEQ. ID. NO. 4)
CAAGCAGAAGACGGCATACnAnAT

where “n” is 8-oxoguanine in each of these sequences.

The P15 primer is:

P15: 5′ → 3′
(SEQ. ID. NO. 5)
AATGATACGGCGACCACCGAGAnCTACAC

where “n” is allyl-T (a thymine nucleotide analog having an allyl functionality).

The other primers (PA-PD) mentioned above include:

PA 5′ → 3′
(SEQ. ID. NO. 6)
GCTGGCACGTCCGAACGCTTCGTTAATCCGTTGAG
PB 5′ → 3′
(SEQ. ID. NO. 7)
CGTCGTCTGCCATGGCGCTTCGGTGGATATGAACT
PC 5′ → 3′
(SEQ. ID. NO. 8)
ACGGCCGCTAATATCAACGCGTCGAATCCGCAACT
PD 5′ → 3′
(SEQ. ID. NO. 9)
GCCGCGTTACGTTAGCCGGACTATTCGATGCAGC

While not shown in the example sequences for PA-PD, it is to be understood that any of these primers may include a cleavage site, such as uracil, 8-oxoguanine, allyl-T, etc. at any point in the strand, as long as the cleavage sites of the primers 20A and 20B are orthogonal (i.e., the cleaving chemistry of the primer 20A is different than the cleaving chemistry for the primer 20B, and thus the two primers 20A, 20B are susceptible to different cleaving agents).

Each of the primers 20A, 20B disclosed herein may also include a polyT sequence at the 5′ end of the primer sequence. In some examples, the polyT region includes from 2 T bases to 20 T bases. As specific examples, the polyT region may include 3, 4, 5, 6, 7, or 10 T bases.

The immobilization of the primers 20A, 20B may be by single point covalent attachment at the 5′ end of the primers 20A, 20B. The 5′ terminal end of the primers 20A, 20B will vary depending upon the chemistry, e.g., functional group 50′, at the surface of the glycolipid bi-layer 18. As two examples, the 5′ end functional groups may be a terminal alkyne (e.g., hexynyl) or an internal alkyne, where the alkyne is part of a cyclic compound (e.g., bicyclo[6.1.0]nonyne (BCN)). The terminal alkynes can attach to azide groups at the surface of the glycolipid bi-layer 18. In another example, the primers 20A, 20B may include an alkene at the 5′ terminus, which can react with reactive thiol groups at the surface of the glycolipid bi-layer 18. In still other specific examples, succinimidyl (NHS) ester terminated primers may be reacted with amine groups at the surface of the glycolipid bi-layer 18, aldehyde terminated primers may be reacted with hydrazine groups at the surface of the glycolipid bi-layer 18, azide terminated primers may be reacted with an alkyne or DBCO (dibenzocyclooctyne) at the surface of the glycolipid bi-layer 18, or amino terminated primers may be reacted with activated carboxylate groups at the surface of the glycolipid bi-layer 18.

Examples of methods for grafting the primers 20A, 20B to the glycolipid bi-layer 18 are described below in reference to FIG. 2 through FIG. 4.

### Additional Components of the Flow Cells

While not shown, it is to be understood that some examples of the flow cells 12A, 12B may further include a lid or a second substrate attached to the single layer substrate 22 or to the multi-layered substrate 28. As described herein, some or all of the interstitial regions 26, 26′ can function as a bonding region where the lid or second substrate will bond to the substrate 22, 28. Once bonded, an enclosed flow cell is formed, which has a flow channel defined between the bonded structures. Other examples of the flow cell 12A, 12B may be open wafers, where the channel and surface chemistry are exposed to the external environment.

When included, the lid may be any material that is transparent to the excitation light that is directed toward the flow cell 12A, 12B. In optical detection systems, the lid may also be transparent to the emissions generated from reaction(s) taking place in the flow cell 12A, 12B. As examples, the lid may include glass (e.g., borosilicate, fused silica, etc.) or a transparent polymer. A commercially available example of a suitable borosilicate glass is D 263®, available from Schott North America Inc. Commercially available examples of suitable polymer materials, namely cyclo olefin polymers, are the ZEONOR® products available from Zeon Chemicals L.P.

The two substrates 22 or 28 or the one substrate 22 or 28 and the lid may be attached to one another through a spacer layer (not shown). The spacer layer may be any material that will seal portions of the substrates 22 or 28 together or portions of the substrate 22 or 28 and the lid. As examples, the spacer layer may be an adhesive, a radiation-absorbing material that aids in bonding, or the like. In some examples, the spacer layer is the radiation-absorbing material, e.g., KAPTON® black. The substrates 22 or 28 or the substrate 22 or 28 and the lid may be bonded using any suitable technique, such as laser bonding, diffusion bonding, anodic bonding, eutectic bonding, plasma activation bonding, glass frit bonding, or others methods known in the art.

### Additional Components of the Functionalized Nanostructures

The functionalized nanostructure 14 (one example of the nucleic acid sequencing component 10C) is also capable of anchoring to a capture site on a flow cell substrate (see FIG. 5A through FIG. 5C). As such, the functionalized nanostructures 14 include some mechanism that is capable of attaching to the capture site. The mechanism may be chemical (e.g., a functional agent), electrostatic, or magnetic.

In some examples, the mechanism is a component of the functionalized nanostructure 14 that enables it to be anchored without further functionalization. For example, when the nanostructure core 38 includes a magnetic material as the mechanism, the functionalized nanostructure 14 may be anchored to a magnetic capture agent on the flow cell substrate. For another example, when the charged amplicons of a pre-clustered functionalized nanostructure is used as the mechanism, the pre-clustered functionalized nanostructure may be anchored to an electrostatic capture agent on the flow cell substrate. The pre-clustered nanostructure is a functionalized nanostructure 14 that has been exposed to cluster generation and thus has a plurality of amplicons attached to the primers 20A, 20B. For still another example, when the glycolipid includes a reversibly chargeable functional group as the mechanism, the functionalized nanostructure 14 may be anchored to an electrostatic capture agent on the flow cell substrate.

In other examples, the mechanism is a functional agent that is added to the functionalized nanostructure 14 that enables it to be anchored on the flow cell substrate. As one example, a target nucleic acid that is complementary to a capture oligonucleotide on the flow cell substrate may be grafted to the glycolipid bi-layer 18. As other examples, the glycolipid bi-layer 18 may include a functional group for covalent attachment to the chemical capture site or a member of a binding pair, the other member of which makes up the chemical capture site.

### Methods for Making the Nucleic Acid Sequencing Component

The nucleic acid sequencing components 10A, 10B, 10C can be formed by a variety a methods.

When preparing the flow cell 12A, the lane 24 may first be defined in the single layer substrate 22 by etching, lithography, or some other suitable technique.

When preparing the flow cell 12B, the material for the patterned layer 32 may be applied to the base 30 in a pattern that defines the depressions 34, or the material for the patterned layer 32 may be applied to the base 30 and then patterned to define the depressions 34. In an example, the patterned layer 32 is an inorganic oxide that is selectively applied to the base 30, e.g., via vapor deposition, aerosol printing, or inkjet printing, in the desired pattern. In another example, the patterned layer 32 is a resin that is deposited on the base 30 and then patterned. Suitable deposition techniques include chemical vapor deposition, dip coating, dunk coating, spin coating, spray coating, puddle dispensing, ultrasonic spray coating, doctor blade coating, aerosol printing, screen printing, microcontact printing, etc. Suitable patterning techniques include photolithography, nanoimprint lithography (NIL), stamping techniques, embossing techniques, molding techniques, microetching techniques, printing techniques, etc. In one example, nanoimprint lithography is used to define the depressions 34.

When preparing the functionalized nanostructure 14, the nanostructure core 38 may be prepared using any suitable method that results in the desired nanostructure.

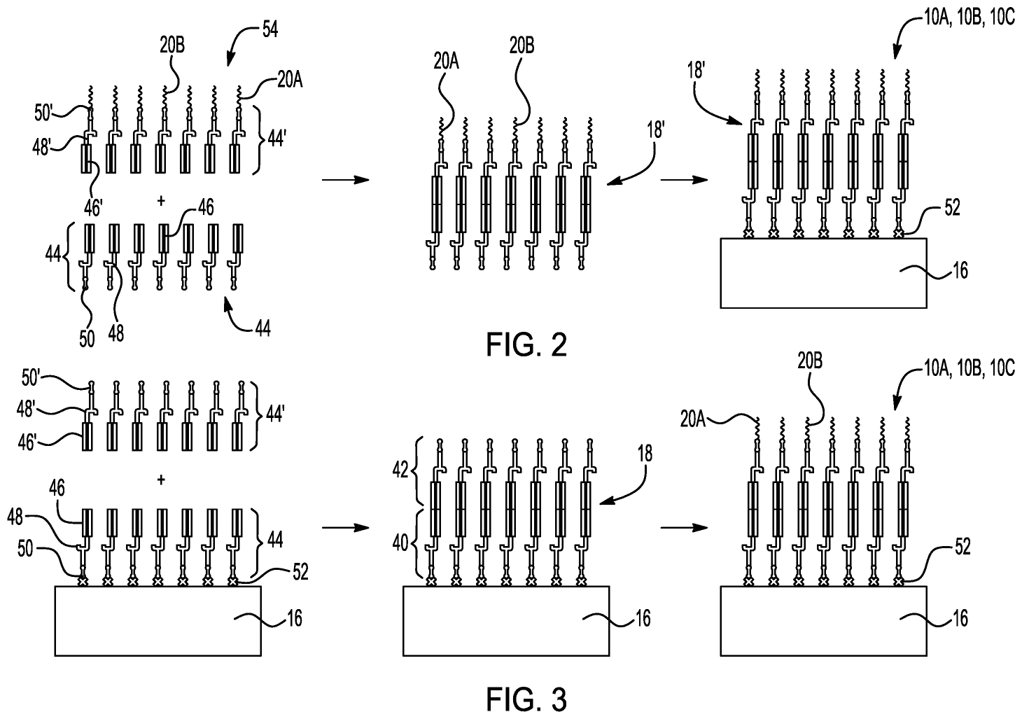

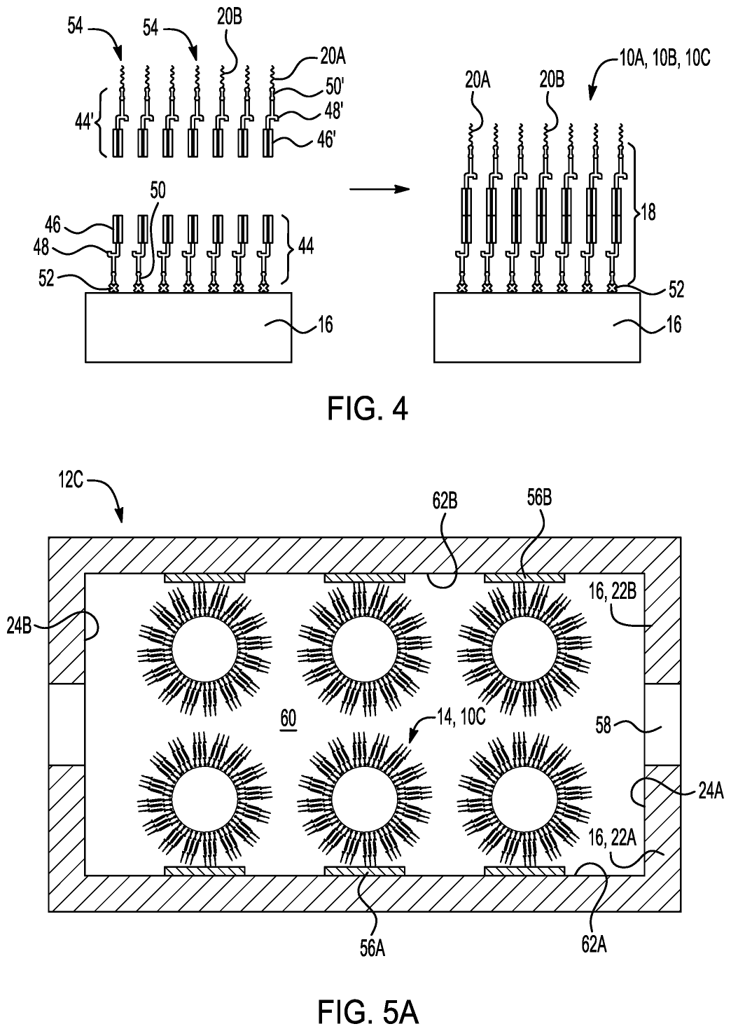

In examples of the nucleic acid sequencing components 10A, 10B, 10C that do not include the polymeric hydrogel 36, the method continues with forming the glycolipid bi-layer 18 so that it is attached to the support 16. Different methods that can be used to form the glycolipid bi-layer 18 are shown in FIG. 2 through FIG. 4. The method shown in FIG. 2 is suitable for use when the support 16 is the substrate 22, 28 or the core nanostructure 38. The methods shown in FIG. 3 and FIG. 4 are suitable for use when the support 16 is the substrate 22, 28. The methods in FIG. 3 and FIG. 4 can lead to aggregation and precipitation of the core nanostructure 38, and thus are not desirable for forming the nucleic acid sequencing components 10C.

The method shown in FIG. 2 includes respectively attaching first and second primers 20A, 20B to at least some of a plurality of first glycolipids 44′; in the presence of water, combining the plurality of first glycolipids 44′ with a plurality of second glycolipids 44, thereby forming a pre-grafted glycolipid bi-layer 18′; and attaching the pre-grafted glycolipid bi-layer 18′ to a support 16 through at least some of the plurality of second glycolipids 44.

The first glycolipids 44′ may be any of the examples described herein in reference to FIG. 1A, FIG. 1B, and FIG. 1C, which include the hydrophobic tail 46′, the sugar group 48′, and the functional group 50′. The glycolipids 44′ and the primers 20A, 20B may be incorporated into a solution or mixture containing water, a buffer, and a catalyst. For carbonate-type grafting, the grafting process may be performed at a temperature ranging from about 55° C. to about 65° C. for a time ranging from about 20 minutes to about 90 minutes. Copper free grafting techniques (e.g., using dibenzocyclooctyne) may also be used, which take place at about 30° C. for about 30 minutes. Bi-cyclo[6.1.0.nonyne (BCN)-type grafting techniques may also be used at room temperature for about 2 hours. The 5′ terminal end of the primers 20A, 20B attaches to the functional group 50′ to form pre-grafted glycolipids 54, as shown in the left portion of FIG. 2.

The pre-grafted glycolipids 54 are then mixed with the second glycolipids 44. The second glycolipids 44 may be any of the examples described herein in reference to FIG. 1A, FIG. 1B, and FIG. 1C, which include the hydrophobic tail 46, the sugar group 48, and the functional group 50. The pre-grafted glycolipids 54 and the glycolipids 44 are mixed in the presence of water. In the presence of water, the pre-grafted glycolipids 54 and the glycolipids 44 self-assemble their hydrophobic tails 46, 46′ in the energetically most favorable manner, resulting in the pre-grafted glycolipid bi-layer 18′ with the hydrophobic tails 46, 46′ aligned at the core of the layer 18′. This is shown in the middle portion of FIG. 2.

The pre-grafted glycolipid bi-layer 18′ is then attached to the support 16 (e.g., 22, 24, or 38), as shown at the right portion of FIG. 2. If the support material does not include the surface groups 52 to attach to the pre-grafted glycolipid bi-layer 18′, it may first be functionalized with the groups 52. For example, the support 16 may be plasma ashed or silanized to introduce the groups 52 to attach to the end group of the functional group 50.

For the substrate supports 16, 22 or 16, 28, the pre-grafted glycolipid bi-layer 18′ may be blanketly deposited over the substrate 16, 22 or 16, 28 (including in the lane 24 or the depressions 34 and on the interstitial regions 26 or 26′), and then removed from the interstitial regions 26 or 26′ using a polishing technique. Alternatively, the pre-grafted glycolipid bi-layer 18′ may be selectively deposited (using a mask to cover interstitial regions 26 or 26′, controlled printing techniques, etc.) to specifically deposit the pre-grafted glycolipid bi-layer 18′ in the lane 24 or in the depressions 34 and not on the interstitial regions 26 or 26′. The conditions used to deposit the pre-grafted glycolipid bi-layer 18′ should not disrupt the bi-layer 18′, and thus should involve aqueous, non-extreme pH, and low ionic strength solutions.

For the core nanostructure supports 16, 38, the pre-grafted glycolipid bi-layer 18′ may be coated onto the surface of the core nanostructure 38. Any suitable deposition technique may be used, such as dip coating, dunk coating, spin coating, spray coating, puddle dispensing, ultrasonic spray coating, etc. In an example, a plurality of core nanostructures 38 may be suspended in a mixture containing the pre-grafted glycolipid bi-layer 18′, and the mixture may be exposed to conditions (e.g., heat) that will initiate the attachment of the pre-grafted glycolipid bi-layer 18′ to the core nanostructure 38.

The methods shown in FIG. 3 and in FIG. 4 generally include attaching a plurality of first glycolipids 44 to at least a portion of a support 16 (in these examples, either of the flow cell substrates 22 or 28); exposing the support to a plurality of second glycolipids 44′ in the presence of water, whereby the plurality of second glycolipids 44′ self-assemble with the plurality of first glycolipids 44 to form a glycolipid bi-layer 18 attached to the support 16; and respectively attaching first and second primers 20A, 20B to at least some of the plurality of second glycolipids 44′.

In both the method of FIG. 3 and the method of FIG. 4, the glycolipids 44 are attached to the support 16 before the glycolipid bi-layer 18 is formed (as shown at the left portion in each of these figures). It is to be understood that if the support material does not include the surface groups 52 to attach to the glycolipids 44 (e.g., through groups 50), the support 16 may first be functionalized with the groups 52. For example, the support 16 may be plasma ashed or silanized to introduce the groups 52 to attach to the functional group 50.

Any of the substrate supports 16 (22 or 28) may be exposed to a mixture containing the glycolipids 44, and then to conditions (e.g., aqueous, relatively neutral pH, and low ionic strength similar to grafting) that will initiate the attachment of the glycolipids 44 to the groups 52 at the surface of the support 16. This forms the surface-bound portion 40 of the glycolipid bi-layer 18.

In the example shown in FIG. 3, the glycolipids 44′ (which include the hydrophobic tail 46′, the sugar group 48′, and the functional group 50′) are exposed to the surface-bound glycolipids 44 (i.e., to portion 40). The glycolipids 44′ may be exposed to the surface-bound glycolipids 44 in the presence of water or a buffer in the presence of salt ions, such as Mg²⁺, Na⁺), which allows the hydrophobic tails 46′ of the glycolipids 44′ to self-assemble with the hydrophobic tails 46 of the surface-bound glycolipids 44. The rate of the reaction is driven primarily by diffusion, and thus an incubation time ranging from about 1 minute to about 2 minutes may be suitable. This forms the glycolipid bi-layer 18, which is attached to the support 16. This is shown in the middle portion of FIG. 3.

As shown in FIG. 3, the first and second primers 20A, 20B are respectively attached to at least some of the plurality of second glycolipids 44′ after the after the glycolipid bi-layer 18 is formed. The primers 20A, 20B may be incorporated into a solution or mixture containing water, a buffer, and a catalyst. The aqueous conditions used for grafting will not disrupt the glycolipid bi-layer 18. The support 16 having the glycolipid bi-layer 18 thereon is then exposed to the primer solution/mixture. For carbonate-type grafting, the grafting process may be performed at a temperature ranging from about 55° C. to about 65° C. for a time ranging from about 20 minutes to about 90 minutes. Copper free grafting techniques (e.g., using dibenzocyclooctyne) may also be used, which take place at about 30° C. for about 30 minutes. Bi-cyclo[6.1.0.nonyne (BCN)-type grafting techniques may also be used at room temperature for about 2 hours. The 5′ terminal end of the primers 20A, 20B attaches to the functional group 50′ to form the grafted glycolipid bi-layer, as shown in the right portion of FIG. 3.

In the example shown in FIG. 4, the first and second primers 20A, 20B are respectively attached to the at least some of the plurality of second glycolipids 44′ before the support 16, and the glycolipids 44 attached thereto, are exposed to the plurality of second glycolipids 44′. In this example, the primers 20A, 20B may be incorporated into a solution or mixture containing water, a buffer, and a catalyst. The glycolipids 44′ are mixed with the primer solution/mixture. In this example, grafting may be performed using any suitable method, as the conditions do not have to take into account disruption of an already formed glycolipid bi-layer 18, 18′. Any of the grafting techniques described herein, as well as other coupling reactions may be used in the method of FIG. 4. As a result of the grafting technique, the 5′ terminal end of the primers 20A, 20B attaches to the functional group 50′ to form the pre-grafted glycolipids 54, as shown in the left portion of FIG. 4.

In this example, the pre-grafted glycolipids 54 are exposed to the surface-bound glycolipids 44 (i.e., to portion 40). The pre-grafted glycolipids 54 may be exposed to the surface-bound glycolipids 44 in the presence of water or a buffer in the presence of salt ions, such as Mg²⁺, Na⁺, which allows the hydrophobic tails 46′ of the pre-grafted glycolipids 54 to self-assemble with the hydrophobic tails 46 of the surface-bound glycolipids 44. This forms the grafted glycolipid bi-layer, which is attached to the support 16. This is shown in the right portion of FIG. 4.

While not shown in FIG. 2 through FIG. 4, it is to be understood that in any of the example methods, the polymeric hydrogel 36 may first be attached to the support 16. Thus, some examples of the methods include attaching a polymeric hydrogel 36 to at least the portion of the support 16 where the glycolipid bi-layer 18 is to be attached, wherein the plurality of first glycolipids 44 is attached to the support 16 through the hydrogel layer 36. The support 16 may be activated (e.g., via plasma ashing or silanization) prior to applying the polymeric hydrogel 36.

A mixture of the polymeric hydrogel 36 may be generated. In one example, the polymeric hydrogel 36 may be present in a mixture (e.g., with water or with ethanol and water).

For the substrate supports 16, 22 or 16, 28, the mixture including the polymeric hydrogel 36 may be blanketly deposited over the substrate 16, 22 or 16, 28 (including in the lane 24 or the depressions 34 and on the interstitial regions 26 or 26′), and then removed from the interstitial regions 26 or 26′ using a polishing technique. Alternatively, the mixture including the polymeric hydrogel 36 may be selectively deposited (using a mask to cover interstitial regions 26 or 26′, controlled printing techniques, etc.) to specifically deposit the polymeric hydrogel 36 in the lane 24 or in the depressions 34 and not on the interstitial regions 26 or 26′.

For the core nanostructure supports 16, 38, the mixture including the polymeric hydrogel 36 may be coated onto the surface of the core nanostructure 38. Any suitable deposition technique may be used, such as dip coating, dunk coating, spin coating, spray coating, puddle dispensing, ultrasonic spray coating, etc. In an example, a plurality of core nanostructures 38 may be suspended in a mixture containing the polymeric hydrogel 36, and the mixture may be exposed to conditions (e.g., heat) that will initiate the attachment of the polymeric hydrogel 36 to the core nanostructure 38.

With any of the supports 16 and depending upon the chemistry of the polymeric hydrogel 36, the applied mixture may be exposed to a curing process. In an example, curing may take place at a temperature ranging from room temperature (e.g., about 25° C.) to about 95° C. for a time ranging from about 1 millisecond to about several days.

### Flow Cells for Use With the Functionalized Nanostructures

The nucleic acid sequencing components 10C shown in FIG. 1C (i.e., the functionalized nanostructure 14) may be used with a flow cell 12C, 12D that includes capture sites 56 (FIG. 5A, FIG. 5B). These examples are shown as enclosed flow cells, where the two substrate 22A, 22B or 28A, 28B (or alternatively, one of the substrate 22, 28 and a lid) are bonded together with a material 58 (e.g., spacer layer). As described herein, this forms a flow channel 60 between the two bonded components. Alternatively, one of the substrates 22A or 28A may be used as an open wafer, without the second substrate 22B or 28B or lid attached thereto.

In the example shown in FIG. 5A, two single layer substrates 22A, 22B are bonded together. Each substrate 22A, 22B has a respective lane 24A, 24B defined therein, which has a substantially flat surface 62A, 62B; and a plurality of capture sites 56A, 56B positioned in respective patterns across each of the substantially flat surfaces 62A, 62B.

The plurality of capture sites 56A, 56B are positioned in respective patterns across the substantially flat surfaces 62A, 62B. The pattern or layout (including density, pitch, etc.) for the capture sites 56A, 56B may be any of the examples set forth herein for the depressions 34.

The capture sites 56A, 56B may have any suitable shape, geometry and dimensions, which may depend, at least in part, on the nucleic acid sequencing components 10C that are to be captured by the capture sites 56A, 56B.

The capture sites 56A, 56B may be chemical capture sites, electrostatic captures sites, or magnetic capture sites.

Chemical capture sites include any example of the chemical capture agent set forth herein that can be deposited on or otherwise attached to predefined locations of the substantially flat surfaces 62A, 62B. In one example, the chemical capture agent may be deposited, e.g., using microcontact printing, aerosol printing, etc., in a desirable location on each substantially flat surface 62A, 62B to form the capture sites 56A, 56B. In another example, a mask (e.g., a photoresist) may be used to define the space/location where the chemical capture agent will be deposited. The chemical capture agent may then be deposited, and the mask removed (e.g., via lift-off, dissolution, or another suitable technique). In this example, the chemical capture agent may form a monolayer or thin layer of the chemical capture agent. In still another example, a polymer grafted with capture nucleic acids may be selectively applied to the substantially flat surfaces 62A, 62B to form the chemical captures sites.

Electrostatic captures sites include any example of the electrostatic capture agents set forth herein that can be deposited on predefined locations of the substantially flat surfaces 62A, 62B. For example, positively charged materials or electrode materials may be deposited using chemical vapor deposition, masking and deposition, or another suitable technique to form the capture sites 56A, 56B. When electrostatic capture sites are used, the substrate 22A, 22B may include additional circuitry to address the individual capture sites 56A, 56B.

Magnetic capture sites include any example of the magnetic capture agent set forth herein that can be deposited on predefined locations of each of the substantially flat surfaces 62A, 62B. For example, magnetic materials may be deposited using chemical vapor deposition, masking and deposition, or another suitable technique to form the capture sites 56A, 56B.

In the example of FIG. 5A, areas of the substantially flat surfaces 62A, 62B that do not contain the capture sites 56A, 56B function as interstitial regions between the capture sites 56A, 56B.

In the examples shown in FIG. 5B, two multi-layered substrate 28A, 28B are bonded together. The substrates 28A, 28B include depressions 34A, 34B separated by interstitial regions 26A, 26B; and a capture site 56C, 56D is positioned in each of the depressions 34A, 34B. The capture sites 56C, 56D may be chemical capture sites, electrostatic captures sites, or magnetic capture sites.

Chemical capture sites include any example of the chemical capture agent set forth herein that can be deposited on or otherwise attached to the bottom surface of each depression 34A, 34B. In one example, the chemical capture agent may be deposited, e.g., using microcontact printing, aerosol printing, etc., on each depression 34A, 34B to form the capture sites 56C, 56D. In another example, a mask (e.g., a photoresist) may be used to cover the interstitial regions 26A, 26B and not the depressions 34A, 34B. The chemical capture agent may then be deposited in the exposed depression 34A, 34B, and the mask removed (e.g., via lift-off, dissolution, or another suitable technique). In this example, the chemical capture agent may form a monolayer or thin layer of the chemical capture agent in the depression 34A, 34B. In still another example, a polymer grafted with capture nucleic acids may be selectively applied to the bottom surface of each depression 34A, 34B.

Electrostatic captures sites include any example of the electrostatic capture agent set forth herein that can be deposited on the bottom surface of each depression 34A, 34B. For example, positively charged or electrode materials may be deposited using chemical vapor deposition, masking and deposition, or another suitable technique to form the capture sites 56C, 56D. When electrostatic capture sites are used, the substrate 28A, 28B may include additional circuitry to address the individual capture sites 56C, 56D.

Magnetic capture sites include any example of the magnetic capture agent set forth herein that can be deposited on the bottom surface of each depression 34A, 34B. For example, magnetic materials may be deposited using chemical vapor deposition, masking and deposition, or another suitable technique to form the capture sites 56C, 56D.

While the examples shown in FIG. 5A and FIG. 5B depict the nucleic acid sequencing components 10C anchored at the captures sites 56A, 56B, 56C, 56D, it is to be understood that the flow cell 12C, 12D does not include the nucleic acid sequencing components 10C until they are introduced thereto, e.g., during sequencing.

In another example of the flow cell that is to be used with the nucleic acid sequencing components 10C, the depressions 34 (shown in FIG. 5B) may instead function as the interstitial regions, and the interstitial regions 26 (shown in FIG. 5B) may be posts that support the capture sites 56. The posts are convex regions with respect to the interstitial regions that surround the posts. In this example, the capture sites 56 may be chemical capture sites, electrostatic captures sites, or magnetic capture sites.

### Kits Including the Functionalized Nanostructures

Any example of the flow cell 12C, 12D and the nucleic acid sequencing component 10C (i.e., the functionalized nanostructure 14) may be part of a kit. An example of the kit includes the flow cell 12C, 12D including a plurality of capture sites 56 and a suspension including a liquid carrier and a plurality of the nucleic acid sequencing components 10C dispersed throughout the liquid carrier. Any example of the nucleic acid sequencing component 10C and any liquid carrier that does not solubilize the nucleic acid sequencing component 10C may be included in the suspension. In the kit, the mechanism of the nucleic acid sequencing component 10C is selected to be able to anchor the nucleic acid sequencing component 10C to the capture site 56 of the flow cell 12C, 12D in the kit.

### Sequencing Method Using the Functionalized Nanostructures

When the nucleic acid sequencing components 10C (i.e., the functionalized nanostructures 14) are to be used in sequencing, they may first be used for the generation of template nucleic acid strands that are to be sequenced.

At the outset of template strand formation, library templates may be prepared from any nucleic acid sample (e.g., a DNA sample or an RNA sample). The DNA nucleic acid sample may be fragmented into single-stranded, similarly sized (e.g., <1000 bp) DNA fragments. The RNA nucleic acid sample may be used to synthesize complementary DNA (cDNA), and the cDNA may be fragmented into single-stranded, similarly sized (e.g., <1000 bp) cDNA fragments. During preparation, adapters may be added to the ends of any of the fragments. Through reduced cycle amplification, different motifs may be introduced in the adapters, such as sequencing primer binding sites, indices, and regions that are complementary to the primers 20A, 20B on the functionalized nanostructures 14. In some examples, the fragments from a single nucleic acid sample have the same adapters added thereto. The final library templates include the DNA or cDNA fragment and adapters at both ends. The DNA or cDNA fragment represents the portion of the final library template that is to be sequenced.

A plurality of library templates may be introduced to a suspension, which includes the liquid carrier and the functionalized nanostructures 14 disclosed herein. Multiple library templates are hybridized, for example, to one of two types of primers 20A, 20B immobilized to the glycolipid bi-layer 18 of the functionalized nanostructures 14.

Amplification of the template nucleic acid strand(s) on the functionalized nanostructures 14 may be initiated to form functionalized nanostructures 14 with a cluster of the template strands attached thereto. In one example, amplification involves cluster generation. In one example of cluster generation, the library templates are copied from the hybridized primers by 3′ extension using a high-fidelity DNA polymerase. The original library templates are denatured, leaving the copies immobilized all around the functionalized nanostructures 14. Isothermal bridge amplification or some other form of amplification may be used to amplify the immobilized copies. For example, the copied templates loop over to hybridize to an adjacent, complementary primer, and a polymerase copies the copied templates to form double stranded bridges, which are denatured to form two single stranded strands. These two strands loop over and hybridize to adjacent, complementary primers and are extended again to form two new double stranded loops. The process is repeated on each template copy by cycles of isothermal denaturation and amplification to create dense clonal clusters on the functionalized nanostructures 14. Each cluster of double stranded bridges is denatured. In an example, the reverse strand is removed by specific base cleavage, leaving forward template strands. Clustering results in the formation of several template strands immobilized on the functionalized nanostructures 14. This example of clustering is referred to as bridge amplification, and is one example of the amplification that may be performed. It is to be understood that other amplification techniques may be used. The functionalized nanostructure 14 including a cluster of the template strands is referred to as a pre-clustered nanostructure.

The pre-clustered nanostructure may be washed to remove unreacted library templates, etc. and suspended in a fresh carrier liquid.

The suspension including the pre-clustered nanostructures may then be introduced into the flow cell 12C, 12D including the plurality of capture sites 56, whereby at least some of the pre-clustered nanostructure respectively attach to at least some of the capture site 56 (as shown in FIG. 5A and FIG. 5B). As described herein, the pre-clustered nanostructure includes a functional agent, the charged amplicons, or a magnetic material that specifically binds, attaches, or is otherwise attracted (e.g., electrostatically, magnetically, etc.) to the capture site 56. The suspension may be allowed to incubate for a predetermined time to allow the pre-clustered nanostructure to become anchored. When electrostatic capture sites 56 are used, the individual sites 56 may be electrically addressed to move the pre-clustered nanostructure toward individual capture sites 56. In this example, the pre-clustered nanostructures may include a reversibly chargeable functional group that can be converted from a neutral species to a charged species at a suitable pH. The charged species can be generated by adjusting the pH, and then attracted to the electrostatic capture sites 56 that are individually or globally addressed.

A wash cycle may be performed to remove any unanchored pre-clustered nanostructure.

Sequencing primers may then be introduced to the flow cell 12C, 12D. The sequencing primers hybridize to a complementary portion of the sequence of the template strands that are attached to the pre-clustered nanostructure. These sequencing primers render the template strands ready for sequencing.

An incorporation mix including labeled nucleotides may then be introduced into the flow cell 12C, 12D, e.g., via an input port that leads to the flow channel 60 or directly into an open channel 60. In addition to the labeled nucleotides, the incorporation mix may include water, a buffer, and polymerases capable of nucleotide incorporation. When the incorporation mix is introduced into the flow cell 12C, 12D, the mix enters the flow channel 60, and contacts the anchored and sequence ready pre-clustered nanostructures.

The incorporation mix is allowed to incubate in the flow cell 12C, 12D, and labeled nucleotides (including optical labels) are incorporated by respective polymerases into the nascent strands along the template strands/amplicons on each of the pre-clustered nanostructures. During incorporation, one of the labeled nucleotides is incorporated, by a respective polymerase, into one nascent strand that extends one sequencing primer and that is complementary to one of the template strands. Incorporation is performed in a template strand dependent fashion, and thus detection of the order and type of labeled nucleotides added to the nascent strand can be used to determine the sequence of the template strand. Incorporation occurs in at least some of the template strands across the pre-clustered nanostructure during a single sequencing cycle.

The incorporated labeled nucleotides may include a reversible termination property due to the presence of a 3′ OH blocking group, which terminates further sequencing primer extension once the labeled nucleotide has been added. After a desired time for incubation and incorporation, the incorporation mix, including non-incorporated labeled nucleotides, may be removed from the flow cell 12C, 12D during a wash cycle. With an enclosed flow cell, the wash cycle may involve a flow-through technique, where a washing solution (e.g., buffer) is directed into, through, and then out of flow channel 60, e.g., by a pump or other suitable mechanism.

Without further incorporation taking place, the most recently incorporated labeled nucleotides can be detected through an imaging event. During the imaging event, an illumination system may provide an excitation light to the flow cell 12C, 12D. The optical labels of the incorporated labeled nucleotides emit optical signals in response to the excitation light.

After imaging is performed, a cleavage mix may then be introduced into the flow cell 12C, 12D. In an example, the cleavage mix is capable of i) removing the 3′ OH blocking group from the incorporated nucleotides, and ii) cleaving the optical label from the incorporated nucleotide. Examples of 3′ OH blocking groups and suitable de-blocking agents/components in the cleavage mix may include: ester moieties that can be removed by base hydrolysis; allyl-moieties that can be removed with NaI, chlorotrimethylsilane and Na₂S₂O₃ or with Hg(II) in acetone/water; azidomethyl which can be cleaved with phosphines, such as tris(2-carboxyethyl)phosphine (TCEP) or tri(hydroxypropyl)phosphine (THP); acetals, such as tert-butoxy-ethoxy which can be cleaved with acidic conditions; MOM (—CH₂OCH₃) moieties that can be cleaved with LiBF₄ and CH₃CN/H₂O; 2,4-dinitrobenzene sulfenyl which can be cleaved with nucleophiles such as thiophenol and thiosulfate; tetrahydrofuranyl ether which can be cleaved with Ag(I) or Hg(II); and 3′ phosphate which can be cleaved by phosphatase enzymes (e.g., polynucleotide kinase). Examples of suitable optical label cleaving agents/components in the cleavage mix may include: sodium periodate, which can cleave a vicinal diol; phosphines, such as tris(2-carboxyethyl)phosphine (TCEP) or tris(hydroxypropyl)phosphine (THP), which can cleave azidomethyl linkages; palladium and THP, which can cleave an allyl; bases, which can cleave ester moieties; or any other suitable cleaving agent.

Additional sequencing cycles may then be performed until the template strands are sequenced.

In other sequencing methods, the suspension of functionalized nanoparticles 14 may first be introduced into the flow cell 12C, 12D and exposed to conditions that help to anchor at least some of the functionalized nanoparticles 14 to the capture sites 56. In these examples, the functionalized nanoparticles 14 do not have the cluster of template strands attached thereto. Rather, the library templates are prepared off-flow cell, and then are introduced into the flow cell 12C, 12D for generation and amplification of the template nucleic acid strands on the already anchored functionalized nanoparticles 14. In this example, any unattached library templates are removed from the flow cell 12C, 12D prior to sequencing, and then sequencing may then be performed as described herein.

### Sequencing Method Using the Flow Cells

When the nucleic acid sequencing components 10A, 10B (i.e., the flow cells 12A, 12B) are to be used in sequencing, they may first be used for the generation of template nucleic acid strands that are to be sequenced.

At the outset of template strand formation, library templates may be prepared as described herein. The plurality of library templates may be suspended in a liquid carrier, and introduced into the flow cell 12A, 12B for cluster generation and amplification across the lane 24 or in each depression 34. In the flow cell 12A, multiple library templates are hybridized, for example, to one of two types of primers 20A, 20B immobilized to the glycolipid bi-layer 18 across the lane 24. In the flow cell 12B, respectively library templates are hybridized, for example, to one of two types of primers 20A, 20B immobilized to the glycolipid bi-layer 18 in each depression 34.

Amplification of the template nucleic acid strand(s) in the lane 24 or in the depressions 34 may be initiated to form multiple clusters of the template strands across the glycolipid bi-layer 18 in the lane 24 or to form different clusters of the template strands across the glycolipid bi-layer 18 in each depression 34. Amplification may be performed as described herein. A wash cycle may be performed to remove any non-seeded template strands.

Sequencing primers may then be introduced to the flow cell 12A, 12B. The sequencing primers hybridize to a complementary portion of the sequence of the template strands that are attached to the glycolipid bi-layer 18. These sequencing primers render the template strands/amplicons ready for sequencing.

An incorporation mix including labeled nucleotides may then be introduced into the flow cell 12A, 12B, e.g., via an input port that leads to the flow channel 60 or directly into an open flow channel 60. When the incorporation mix is introduced into the flow cell 12A, 12B, the mix enters the flow channel, and contacts the amplicons.

The incorporation mix is allowed to incubate in the flow cell 12A, 12B, and labeled nucleotides (including optical labels) are incorporated by respective polymerases into the nascent strands along the template strands/amplicons on the glycolipid bi-layer 18. Incorporation occurs in at least some of the template strands/amplicons across the lane 24 or in at least some of the depressions 34 during a single sequencing cycle.

The incorporated labeled nucleotides may include a reversible termination property due to the presence of a 3′ OH blocking group, which terminates further sequencing primer extension once the labeled nucleotide has been added. After a desired time for incubation and incorporation, the incorporation mix, including non-incorporated labeled nucleotides, may be removed from the flow cell 12A, 12B during a wash cycle. In an enclosed flow cell, the wash cycle may involve a flow-through technique, where a washing solution (e.g., buffer) is directed into, through, and then out of flow channel 60, e.g., by a pump or other suitable mechanism.

Without further incorporation taking place, the most recently incorporated labeled nucleotides can be detected through an imaging event. During the imaging event, an illumination system may provide an excitation light to the flow cell 12A, 12B. The optical labels of the incorporated labeled nucleotides emit optical signals in response to the excitation light.

After imaging is performed, a cleavage mix may then be introduced into the flow cell 12A, 12B. Any of the cleavage mixes described herein may be used. Additional sequencing cycles may then be performed until the template strands are sequenced.

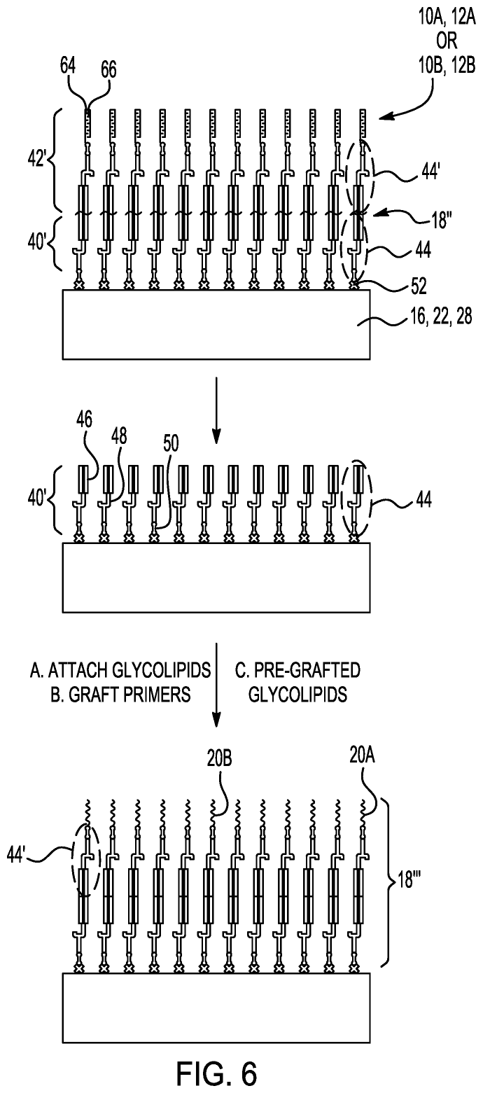

After sequencing is performed in the flow cells 12A, 12B, the sequencing surface can be regenerated. Examples of this method are shown in FIG. 6. These methods involve disrupting the glycolipid bi-layer 18 (which, after sequencing, has the amplicons 64 and nascent strands 66 attached thereto); and removing, from the flow cell 12A, 12B, a portion 42′ of the disrupted glycolipid bi-layer 18″ having the plurality of first and second primers 20A, 20B, the plurality of amplicons 64, and the plurality of nascent strands 66 attached thereto.

Because the glycolipids 44, 44′ of the glycolipid bi-layer 18 are not covalently attached to each other, the glycolipid bi-layer 18 may be disrupted. In one example, disrupting the glycolipid bi-layer 18 involves sonicating the flow cell 12A, 12B. In another example, disrupting the glycolipid bi-layer 18 involves introducing an enzyme into the flow cell 12A, 12B. Suitable enzyme family classes that may be used for glycolipid bi-layer 18 disruption include E.C. 7.6.2.1 flippases, floppases, and scramblases.

The sonication or the enzymes disrupt the glycolipid bi-layer 18 where the hydrophobic tails 46, 46′ are self-assembled, which is schematically illustrated by the jagged lines between the hydrophobic tails 46, 46′ at the top portion of FIG. 6. The portion 42′ of the disrupted glycolipid bi-layer 18″ can be washed away, as it is no longer attached to the support 16. In contrast, the portion 40′ of the disrupted glycolipid bi-layer 18″ remains attached to the support 16. The remaining portion 40′ after washing is shown in the middle portion of FIG. 6.

One example of the method then includes introducing, into the flow cell 12A, 12B, a plurality of glycolipids 44′ that self-assemble with the portion 40′ of the disrupted glycolipid bi-layer 18″ (i.e., the portion 40′ that remains attached to the support 16), thereby generating a fresh glycolipid bi-layer 18′“; and grafting fresh first and second primers 20A, 20B to the fresh glycolipid bi-layer 18”'. The self-assembly of the glycolipids 44′ followed by the primer 20A, 20B grafting may be performed as described in reference to FIG. 3.

Another example of the method then includes introducing, into the flow cell 12A, 12B, a plurality of pre-grafted glycolipids 54 that self-assemble with the second portion 40′ of the disrupted glycolipid bi-layer 18″, thereby generating a fresh glycolipid bi-layer 18′″ having fresh first and second primers 20A, 20B attached thereto. The pre-grafted glycolipids 54 may be prepared as described in reference to FIG. 2.

The bottom portion of FIG. 6 illustrates the regenerated glycolipid bi-layer 18′″ with the primers 20A, 20B that can be exposed to amplification (cluster generation to generate amplicons for another sequencing operation.

### Additional Notes

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

Reference throughout the specification to “one example”, “another example”, “an example”, and so forth, means that a particular element (e.g., feature, structure, and/or characteristic) described in connection with the example is included in at least one example described herein, and may or may not be present in other examples. In addition, it is to be understood that the described elements for any example may be combined in any suitable manner in the various examples unless the context clearly dictates otherwise.

While several examples have been described in detail, it is to be understood that the disclosed examples may be modified. Therefore, the foregoing description is to be considered non-limiting.

What is claimed is:

1. A nucleic acid sequencing component, comprising:
a support;
a glycolipid bi-layer attached to at least a portion of the support; and
first and second primers respectively attached to the glycolipid bi-layer.

2. The nucleic acid sequencing component as defined in claim 1, wherein:
the glycolipid bi-layer includes:
a first portion including first glycolipid residues attached to the portion of the support, wherein each of the first glycolipid residues includes a first hydrophobic tail and a first hydrophilic sugar group; and
a second portion including second glycolipid residues self-assembled with the first lipid residues, wherein each of the second glycolipid residues includes a second hydrophobic tail and a second hydrophilic sugar group; and
the first and second primers are respectively attached to at least some of the second hydrophilic sugar groups.

3. The nucleic acid sequencing component as defined in claim 2, wherein the first hydrophilic sugar group and the second hydrophilic sugar group are independently selected from the group consisting of a six-carbon sugar, a five-carbon sugar, a four-carbon sugar, a seven-carbon sugar, a deoxysugar, a di-deoxysugar, an acidic sugar, a sugar alcohol, and combinations thereof, and wherein the first hydrophobic tail and the second hydrophobic tail are independently selected from the group consisting of a bacterial phospholipid and an archaeal lipid.

4. The nucleic acid sequencing component as defined in claim 1, wherein:
the support is a substrate;
the nucleic acid sequencing component further comprises a hydrogel layer attached to at least a portion of the substrate; and
the glycolipid bi-layer is attached to the hydrogel layer.

5. The nucleic acid sequencing component as defined in claim 1, wherein the support is glass and the glycolipid bi-layer is attached to the glass.

6. The nucleic acid sequencing component as defined in claim 1, wherein:
the support is a substrate that includes depressions surrounded by interstitial regions; and
the glycolipid bi-layer and the first and second primers are positioned within at least some of the depressions.

7. The nucleic acid sequencing component as defined in claim 1 wherein:
the support is a substrate that includes a lane surrounded by bonding regions; and
the glycolipid bi-layer and the first and second primers are positioned within the lane.

8. The nucleic acid sequencing component as defined in claim 1 wherein the first and second primers are terminated with functional groups that covalently attach to functionalized hydrophilic sugar groups of the glycolipid bi-layer.

9. The nucleic acid sequencing component as defined in claim 1, wherein the support is a core nanostructure.

10. The nucleic acid sequencing component as defined in claim 9, further comprising a hydrogel layer attached to the core nanostructure, wherein the glycolipid bi-layer is attached to the hydrogel layer.

11. The nucleic acid sequencing component as defined in claim 1, wherein the nucleic acid sequencing component is a flow cell that includes a lid attached to the support or a second nucleic acid sequencing component attached to the support.

12. A method, comprising:
attaching a plurality of first glycolipids to at least a portion of a support;
exposing the support to a plurality of second glycolipids in the presence of water, whereby the plurality of second glycolipids self-assemble with the plurality of first glycolipids to form a glycolipid bi-layer attached to the support; and
respectively attaching first and second primers to at least some of the plurality of second glycolipids.

13. The method as defined in claim 12, wherein the first and second primers are respectively attached to the at least some of the plurality of second glycolipids before the support is exposed to the plurality of second glycolipids.

14. The method as defined in claim 12, wherein the first and second primers are respectively attached to the at least some of the plurality of second glycolipids after the glycolipid bi-layer is formed.

15. The method as defined in claim 12, wherein the support is selected from the group consisting of a substrate and a core nanostructure.

16. The method as defined in claim 12, further comprising attaching a hydrogel layer to the at least the portion of the support, wherein the plurality of first glycolipids is attached to the support through the hydrogel layer.

17. A method, comprising:
respectively attaching first and second primers to at least some of a plurality of first glycolipids;
in the presence of water, combining the plurality of first glycolipids with a plurality of second glycolipids, thereby forming a pre-grafted glycolipid bi-layer; and
attaching the pre-grafted glycolipid bi-layer to a support through at least some of the plurality of second glycolipids.

18. The method as defined in claim 17, wherein the support is selected from the group consisting of a substrate and a core nanostructure.

19. The method as defined in claim 17, further comprising attaching a hydrogel layer to the at least the portion of the support, wherein the plurality of first glycolipids is attached to the support through the hydrogel layer.

20. A method comprising:
amplifying a plurality of library template strands on a surface of a flow cell to generate a plurality of amplicons, the flow cell including:
a support;
a glycolipid bi-layer attached to at least a portion of the support; and
first and second primers respectively attached to the glycolipid bi-layer, wherein each of the plurality of amplicons is attached to a respective one of the plurality of first and second primers;
performing a sequencing operation involving the plurality of amplicons, thereby generating a plurality of nascent strands respectively attached to the plurality of amplicons;
disrupting the glycolipid bi-layer; and
removing, from the flow cell, a portion of the disrupted glycolipid bi-layer having the plurality of first and second primers, the plurality of amplicons, and the plurality of nascent strands attached thereto.

21. The method as defined in claim 20, wherein disrupting the glycolipid bi-layer involves sonicating the flow cell.

22. The method as defined in claim 20, wherein disrupting the glycolipid bi-layer involves introducing an enzyme into the flow cell.

23. The method as defined in claim 20, wherein a second portion of the glycolipid bi-layer remains attached to the at least the portion of the support, and the method further comprises introducing, into the flow cell, a plurality of pre-grafted glycolipids that self-assemble with the second portion of the disrupted glycolipid bi-layer, thereby generating a fresh glycolipid bi-layer having fresh first and second primers attached thereto.

24. The method as defined in claim 20, wherein a second portion of the glycolipid bi-layer remains attached to the at least the portion of the support, and the method further comprises:
introducing, into the flow cell, a plurality of glycolipids that self-assemble with the second portion of the disrupted glycolipid bi-layer, thereby generating a fresh glycolipid bi-layer; and
grafting fresh first and second primers to the fresh glycolipid bi-layer.


SEQUENCE LISTING (9 sequences)

SEQ ID NO: 1 - DNA, 29 bp, synthetic construct
  source 1..29: mol_type = other DNA
  modified_base 23: mod_base = OTHER; note = URACIL
aatgatacggcgaccaccgagatctacac

SEQ ID NO: 2 - DNA, 24 bp, synthetic construct
  source 1..24: mol_type = other DNA
  modified_base 22: mod_base = OTHER; note = 8-oxoguanine
caagcagaagacggcatacganat

SEQ ID NO: 3 - DNA, 24 bp, synthetic construct
  source 1..24: mol_type = other DNA
  modified_base 20: mod_base = OTHER; note = 8-oxoguanine
caagcagaagacggcatacnagat

SEQ ID NO: 4 - DNA, 24 bp, synthetic construct
  source 1..24: mol_type = other DNA
  modified_base 20: mod_base = OTHER; note = 8-oxoguanine
  modified_base 22: mod_base = OTHER; note = 8-oxoguanine
caagcagaagacggcatacnanat

SEQ ID NO: 5 - DNA, 29 bp, synthetic construct
  source 1..29: mol_type = other DNA
  modified_base 23: mod_base = OTHER; note = allyl-T
aatgatacggcgaccaccgaganctacac

SEQ ID NO: 6 - DNA, 35 bp, synthetic construct
  source 1..35: mol_type = other DNA
gctggcacgtccgaacgcttcgttaatccgttgag

SEQ ID NO: 7 - DNA, 35 bp, synthetic construct
  source 1..35: mol_type = other DNA
cgtcgtctgccatggcgcttcggtggatatgaact

SEQ ID NO: 8 - DNA, 35 bp, synthetic construct
  source 1..35: mol_type = other DNA
acggccgctaatatcaacgcgtcgaatccgcaact

SEQ ID NO: 9 - DNA, 34 bp, synthetic construct
  source 1..34: mol_type = other DNA
gccgcgttacgttagccggactattcgatgcagc